(19) United States
(12) Patent Application Publication
(10) Pub. No.: US 2018/0214382 A1
(43) Pub. Date: Aug. 2, 2018

(54) EDIBLE COMPOSITION COMPRISING A POLYSACCHARIDE AND A LIPID

(71) Applicant: PERORA GMBH, Heidelberg (DE)

(72) Inventor: Dirk VETTER, Heidelberg (DE)

(21) Appl. No.: 15/742,382

(22) PCT Filed: Jul. 7, 2016
(86) PCT No.: PCT/EP2016/066220

§ 371 Date: Jan. 5, 2018

(30) Foreign Application Priority Data
Jul. 7, 2015 (EP) 15175571.7
Jul. 8, 2015 (EP) 15175819.0
Aug. 11, 2015 (EP) 15180659.3
Dec. 23, 2015 (EP) 15202552.4

(51) Int. Cl. A61K 9/20; A61K 31/23; A61K 9/00; A61P 3/04; A23L 33/21; A23L 7/126

(52) U.S. Cl. CPC A61K 9/2059; A61K 9/205; A61K 31/23; A61K 9/2095; A23V 2002/00; A61P 3/04; A23L 33/21; A23L 7/126; A61K 9/0056

(57) ABSTRACT

The invention provides ingestible solid phase comprising an intimate mixture of a water-soluble or water-insoluble polysaccharide component (e.g. dietary fibres) and a fatty acid glyceride component having a melting point of higher than 37° C.; this solid phase optionally being provided in particulate form. The invention further provides methods for preparing the ingestible solid phase and uses thereof.

### FIELD

The present invention relates to oral compositions for the delivery of bioactive agents to the gastrointestinal tract.

### BACKGROUND

In the field of oral drug delivery gastroretentive dosage forms have been developed for a number of bioactive substances, typically synthetic compounds, so called small molecules. Often such synthetic compounds require a slow release from their dosage form after oral administration to minimise side effects and maximise efficacy. For this purpose drug substances may be incorporated in a matrix comprising lipids, sometimes in combination with mucoadhesive polymers in order to bind to the mucosa of the gastric wall and prolong the residence time of the system. For instance, U.S. 2006/0134144 details mucoadhesive compositions for solubilisation of insoluble drugs, formulated with monoglycerides and oil. EP 0580861 to Nippon Shinyaku Company claims a sustained release capsule for adhesion in the gastrointestinal tract filled with drug substance, adhesion polymer and filler polymers and liquid paraffin. U.S. Pat. No. 5,571,533 to Recordati discloses controlled-release furosemide-lipid granules coated with mucoadhesive polymers. U.S. Pat. No. 6,368,635 to Takeda Chemical Industries describes mucoadhesive matrices, prepared by mixing high-melting triglyceride with drug substance and acryl acid polymer. From recent research in the area of anti-obesity, though, it has emerged that triglycerides or their digestive degradation products, free fatty acids, may act as bioactive substances in their own right. For instance, it is well documented that the infusion of lauric acid or oleic acid into the duodenum by means of a feeding tube provides for strong satiety signalling, which may be useful in the treatment and/or prevention of obesity. Consequently, there is a need to provide sustained release formulations of free fatty acids.

Numerous invasive methods and systems are known for use in the treatment and/or prevention of obesity. For instance, WO 2011/136975 Al describes gastric bands, and in particular a method and system for displaying gastric band information which can support adjustment of the gastric band. The adjustment of the gastric band may be dependent on several pieces of data such as satiety state data.

Alternative non-invasive approaches for the treatment of obesity may infer satiety or the feeling of fullness or satisfaction through a variety of different ingestible compositions such as gelling systems, swelling or expandable systems or certain nutrient compositions.

It is an object of the present invention to provide an effective method for delivering fatty acids and lipids based on fatty acids to the gastrointestinal tract. A further object is to provide means for the delivering such fatty acids and lipids to specific regions within the gastrointestinal tract, such as the stomach or the duodenum. A further object is to provide compositions, dosage forms and/or formulations which are useful for the oral delivery of fatty acids and lipids based on fatty acids. A yet further object is to provide a method for the treatment and/or prevention of obesity and diseases or conditions associated with obesity.

Further objects will become apparent on the basis of the following description including the examples, and the patent claims.

### SUMMARY OF THE INVENTION

In a first aspect, the invention provides an ingestible solid phase comprising an intimate mixture of

(a) at least 10 wt.-% of a water-soluble polysaccharide component based on glucose or fructose having an average degree of polymerisation from 2 to 100, or from 4 to 80, and
(b) at least 10 wt.-% of a fatty acid glyceride component having a melting point of higher than 37° C., wherein the solid phase comprises not more than 5 wt.-% of mucoadhesive polymer, and wherein the solid phase is provided in the form of
(i) at least one ingestible particle having a sieve diameter in the range from 0.01 mm to 10 mm, or from 0.01 mm to 20 mm; or
(ii) an ingestible non-particulate matrix.

In another aspect, the invention provides an ingestible solid phase comprising an intimate mixture of

(a) at least 10 wt.-% of a water-soluble polysaccharide component based on glucose or fructose having an average degree of polymerisation from 2 to 100, or from 4 to 80, and
(b) at least 10 wt.-% of a fatty acid glyceride component having a melting point of higher than 37° C., wherein the combined content of the water-soluble polysaccharide component and of the fatty acid glyceride component in the solid phase is at least 80 wt.-%, and wherein the solid phase is provided in the form of
(i) at least one ingestible particle having a sieve diameter in the range from 0.01 mm to 10 mm, or from 0.01 mm to 20 mm; or
(ii) an ingestible non-particulate matrix.

In an alternative embodiment of the invention, the water-soluble polysaccharide component in the ingestible solid phase as described above may be replaced by a water-insoluble, non-swelling, edible polysaccharide component, preferably a polysaccharide component which is resistant to digestion in the human small intestine, such as cellulose, hemicellulose, or long-chain and/or branched beta-glucans like curdlan.

In this case, the inventive solid phase may comprise an intimate mixture of

(a) at least 10 wt.-% of a water-insoluble, non-swelling, edible polysaccharide component, and
(b) at least 10 wt.-% of a fatty acid glyceride component having a melting point of higher than 37° C., wherein the solid phase comprises not more than 5 wt.-% of mucoadhesive polymer, and/or wherein the combined content of the water-insoluble, non-swelling, edible polysaccharide component and of the fatty acid glyceride component in the solid phase is at least 80 wt.-%, and wherein the solid phase is provided in the form of
(i) at least one ingestible particle having a sieve diameter in the range from 0.01 mm to 10 mm, or from 0.01 mm to 20 mm; or
(ii) an ingestible non-particulate matrix.

The solid phase in its particulate form, i.e. in form of the ingestible particles, may be orally administered as such, or blended with comestible goods, including drinks, prior to consumption; said blends optionally being provided to the consumer in a ‘ready-to-use’ form.

In an alternative aspect, the solid phase may be applied directly to and/or blended with comestible goods, without forming particles of it first, i.e. in the form of an ingestible non-particulate matrix. For instance, the mixture may be coated onto comestible goods like cornflakes or puffed rice or puffed soy; it may be applied onto, or in between, layers of crispbread to form sandwich type, ready-to-eat snack bars; or it may be employed as a binder, e.g. blended with cereals such as rolled oats and formed into a muesli bar type food product.

The solid phase may be substantially free of mucoadhesive polymers; in other words, the solid phase may essentially consist of the water-soluble polysaccharide component and the fatty acid glyceride component and optionally one or more further excipients without mucoadhesive properties, such as sugars, sugar alcohols, vitamins, amino acids, proteins and/or micro-nutrients.

The water-soluble polysaccharide component may exhibit a solubility of at least 2 wt.-%, and optionally of at least 5 wt.-%, in purified water at 25° C. The water-soluble or water-insoluble polysaccharide component may comprise a neutral polysaccharide that is resistant to digestion in the human small intestine. Examples of suitable water-soluble polysaccharide components include dextrins, inulins and glucans such as beta-glucans; in particular dextrins having a degree of polymerisation from 2 to 100, preferably from4 to 40 or 5 to 40 or 8 to 40, in particular from 10 to 30 and further preferably from 12 to 25; or inulins having a degree of polymerisation from 2 to 100, preferably or 4 to 60 or 10 to 60, more preferably 3 to 50 or 4 to 30 or from 5 to 25; or soluble cereal-derived beta-glucans like oat—or barley beta-glucan. Examples of suitable water-insoluble, non-swelling, edible polysaccharide components include cellulose, hemicellulose, or long-chain and/or branched beta-glucans like curdlan.

The fatty acid glyceride component may have a melting point from 38° C. to 75° C., in particular from 40° C. to 70° C. The fatty acid glyceride component may comprise fatty acid triglycerides; the content of the fatty acid triglyceride in the solid phase being at least 10 wt.-%.

The solid phase may e.g. comprise a water-soluble polysaccharide component comprising a resistant dextrin derived from wheat or maize starch, and a fatty acid glyceride component comprising a fractionated but non-hydrogenated palm stearin or palm kernel stearin.

The particles according to the invention may be in the form of a granule, a pellet, a minitablet, a flake, a shaving, a chip, a sprinkle or a mixture thereof. The particles according to the invention may have a mass median sieve diameter in the range from 0.01 mm to 10 mm, or even from 0.01 mm to 20 mm.

In a further aspect, the invention provides a method for the preparation of the solid phase according to the invention comprising the steps of (i) preparing an intimate mixture comprising the water-soluble polysaccharide component and the fatty acid glyceride component (for instance by melting the fatty acid glyceride component at least partially and blending in the polysaccharide component), and
(ii) applying the intimate mixture onto comestible goods prior to solidification;
(iii) blending the intimate mixture with comestible goods prior to solidification; or
(iv) processing the intimate mixture further to obtain particles of said mixture by extrusion using a screw extruder; spray congealing; melt granulation; compression into minitablets; melt injection into a liquid medium; or spray coating onto inert cores.

In a further aspect, the invention provides compositions for oral administration which comprise the ingestible solid phase or which are prepared from it, with the solid phase being provided either in the form of

(a) particles (such as pellets in bottles, sachets, stick packs, capsules or tablets or other dosage units; flakes blended into mueslis or ‘trail mix’ compositions; sprinkles added to yoghurt; chips incorporated into snack-or muesli bars; or the like); or
(b) in the form of an ingestible, non-particulate matrix, such as ready-to-eat snack bars formed with the solid phase as a binder as described above. The compositions may comprise comestible goods, including ingestible liquids and semi-solids, which may either be provided combined with the solid phase in a ready-to-use composition, or separately such that the consumer, or user, adds it to the solid phase prior to ingestion.

Single dose units or packages may be provided comprising these solid phase compositions for oral administration, wherein the amount of the composition is from 3 g to 100 g, or from 3 g to 20 g, and/or wherein the amount of the fatty acid glyceride component in the composition is at least 1 g, preferably at least 2 g.

In a yet further aspect, the invention provides the use of the solid phase and of the oral compositions based on it for the prevention and/or treatment of obesity or a disease or condition associated with obesity, for appetite suppression, for the induction of satiety, and/or for body weight reduction. Optionally, the compositions are administered orally at least once a day over a period of at least one week for this purpose.

### DETAILED DESCRIPTION OF THE INVENTION

In a first aspect, the invention provides an ingestible solid phase comprising an intimate mixture of (a) at least 10 wt.-% of a water-soluble polysaccharide component based on glucose or fructose having an average degree of polymerisation from 2 to 100, or from 4 to 80 (hereafter referred to as ‘the polysaccharide component’), and (b) at least 10 wt.-% of a fatty acid glyceride component having a melting point of higher than 37 ° C. (hereafter referred to as ‘the fatty acid glyceride component’), wherein the particle comprises not more than 5 wt.-% of mucoadhesive polymer, wherein the solid phase is provided in the form of
(i) at least one ingestible particle having a sieve diameter in the range from 0.01 mm to 10 mm, or from 0.01 mm to 20 mm; or
(ii) an ingestible non-particulate matrix.

In another aspect, the invention provides an ingestible solid phase comprising an intimate mixture of (a) at least 10 wt.-% of a water-soluble polysaccharide component based on glucose or fructose having an average degree of polymerisation from 2 to 100, or from 4 to 80, and (b) at least 10 wt.-% of a fatty acid glyceride component having a melting point of higher than 37° C., wherein the combined content of the water-soluble polysaccharide component and of the fatty acid glyceride component in the particle is at least 80 wt.-% , wherein the solid phase is provided in the form of
(i) at least one ingestible particle having a sieve diameter in the range from 0.01 mm to 10 mm, or from 0.01 mm to 20 mm; or
(ii) an ingestible non-particulate matrix.

In an alternative aspect of the invention, the water-soluble polysaccharide component in the embodiments described above may be replaced by a water-insoluble, non-swelling, edible polysaccharide component (also referred to hereafter as the ‘polysaccharide component’ unless specified otherwise); like e.g. cellulose, hemicellulose or long-chain and/or branched beta-glucans like curdlan. I.e. in this alternative embodiment, the solid phase comprises an intimate mixture of

(a) at least 10 wt.-% of a water-insoluble, non-swelling, edible polysaccharide component, and
(b) at least 10 wt.-% of a fatty acid glyceride component having a melting point of higher than 37° C., wherein the solid phase comprises not more than 5 wt.-% of mucoadhesive polymer, and/or wherein the combined content of the water-insoluble, non-swelling, edible polysaccharide component and of the fatty acid glyceride component in the solid phase is at least 80 wt.-%, and wherein the solid phase is provided in the form of
(i) at least one ingestible particle having a sieve diameter in the range from 0.01 mm to 10 mm, or from 0.01 mm to 20 mm; or
(ii) an ingestible non-particulate matrix.

Unless specified otherwise, the terms ‘the mixture’ or ‘the intimate mixture’ herein refers to the mixture according to the invention as described above; i.e. intimately mixed blends of the specific polysaccharide component(s) and the specific fatty acid glyceride component(s), with a combined polysaccharide/fatty acid glyceride content of at least 80 wt.-% and not more than 5 wt.-% of mucoadhesive polymer.

According to the invention, the polysaccharide component is in an intimate mixture with the fatty acid glyceride component. This means that one component, usually the polysaccharide component, may be largely dispersed within the other component, usually the fatty acid glyceride component, whether molecularly, colloidally or in the form of a solid suspension; i.e. the polysaccharide component(s) and the fatty acid glyceride component(s) are inseparably blended into one coherent solid phase, wherein it is irrespective how this coherence and/or inseparability are achieved. The fatty acid glyceride component may form a continuous phase in which the polysaccharide component is discontinuous and in dispersed form. For the avoidance of doubt, this does not exclude that some of the polysaccharide component is not fully dispersed, but positioned at the outer surface of the lipid component.

Alternatively—and depending on the specific ratio of the components to each other, as well as the method of forming the mixture—both components may be dispersed by/in each other with no clear distinction between continuous and discontinuous phase. The main requirement of an intimate mixture is that the mixture is relatively homogenous, as will be understood by the skilled person.

Examples of intimate mixtures according to the invention include melts of the fatty acid glyceride component into which the polysaccharide component is mixed prior to cooling down, as well as any co-processed mixture in which one component (commonly the polysaccharide) is embedded in and/or coated with the other (commonly the fatty acid glyceride component). Examples of what is not considered an intimate mixture according to the invention include mere uncompressed powder blends of the two components, because such powder blends may in theory be separated and thereby lose their homogeneity (e.g. one component being wetted by an ingestible liquid, while the other is poorly wetted and may exhibit floatation).

It should further be understood that, as used herein, the terms ‘a’ or ‘an’ or ‘the’ or features described in their singular form do not exclude a plurality of the respective features. Unless explicitly stated or described otherwise, expressions such as “an water-soluble polysaccharide component”, “a fatty acid glyceride component” or the like are chosen solely for reasons of simplicity and are meant to encompass one or more water-soluble polysaccharide component(s), fatty acid glyceride component(s), etc.; e.g. in the form of blends, or mixtures, of two or more of the respective components.

All percentages, parts and ratios as used herein, are by weight of the total formulation, unless otherwise specified; i.e. “%” should be read as “wt.-%” unless otherwise specified.

The solid phase according to the invention may be provided to the consumer, or user in the form of particles (e.g. granules, pellets, minitablets, flakes, shavings, chips, sprinkles or the like), which—depending on their size—may or may not require chewing prior to being swallowed.

Alternatively, the solid phase may also be provided to the consumer as a non-particulate matrix; for instance, in the form of a solidified ‘spread’ or coating on or in between layers of crispbread or wafers, or in the form of a solidified binder in cereal-based muesli bars. In this non-particulate form, the solid phase typically requires chewing by the consumer in order to form smaller, swallowable pieces; said pieces then behaving similar in vivo as the particles of the invention. Further details on the different aspects of the invention are detailed below.

The inventors have found that the ingestible solid phase as defined herein, and in particular oral compositions comprising or prepared from the solid phase, are capable of effectively inducing satiety, of suppressing the appetite, and therefore may be used to prevent and/or treat obesity or conditions associated with obesity; e.g. by using the ingestible solid phase as defined herein and/or compositions comprising or prepared from it for body weight reduction.

Without wishing to be bound by theory, it is currently believed that upon oral administration, the fatty acid or fatty acid ester comprised in the solid phase is more effectively delivered to the mucosa of the gastrointestinal tract, such as the stomach or duodenum, and in particular to the receptors in the upper intestinal mucosa which are involved in the signalling of satiety, by virtue of the polysaccharide component, which may be instrumental in providing a prolonged or otherwise increased interaction of the fatty acid material with target structures at/in the mucosa. Without wishing to be bound by theory, this increase in bioavailability may e.g. be caused by the prolonged integrity of the solid phase particle(s) according to the invention (and/or the masticated pieces of the solid phase), which may be associated with a more rapid gastric emptying of the particle(s)/pieces; similar to the more rapid gastric emptying observed with pellet formulations, which are believed to pass even the closed pylorus.

Possibly, the polysaccharide component prolongs the integrity of the solid phase particles after ingestion as compared to a lipid particle without the polysaccharide component. Prolongation of particle integrity is the prolongation of time during incubation under in vivo or simulated in vivo conditions in which the majority (more than 50%) of particles do not decrease their volume or mass or melt into droplets. Particle integrity may be readily inferred by visual inspection by the naked eye or by means of a microscope or through imaging technology, including microscopic imaging, and subsequent computer-aided image processing. Prolonged integrity of the lipid-containing particle may result in more rapid gastric emptying of the particles and therefore more rapid interaction of particle-derived fatty acids or fatty acid esters with the intestinal mucosa. Prolonged integrity of the lipid-containing particle may also result in the delivery of fatty acids or fatty-acid esters to the more distal parts of the small intestine such as the jejunum or ileum. With regard to particle integrity, it should be understood, that the same considerations typically also apply to the masticated pieces of the solid phase.

Possibly, the polysaccharide component increases the digestibility of a lipid component of otherwise limited digestibility such as tristearin. In a published rat feeding study, tristearin (Dynasan® 118, melting range 72-75° C.) was found to provide an energy content of only 3 kcal/g, corresponding to a true digestibility of stearic acid from tristearin of only 0.15 g/g independent from intake. Possibly, the polysaccharide component enhances the particle's surface wetting properties and/or facilitates water and bile acid access and subsequent emulsification and lipase-mediated hydrolysis of the lipid.

In any case, the inventors have found that the oral administration of the solid phase of the invention to human subjects leads to a sensation of satiety, or increased satiety.

As explained above, the solid phase of the invention may be provided in the form of an ingestible non-particulate matrix or in the form of ingestible particles. As used herein, an ingestible particle is a particle which is in principle suitable for oral ingestion, or oral administration. A particle which by virtue of its composition, size and morphology would be suitable as a food component or a component of a pharmaceutical composition for oral use is an example of an ingestible particle.

The particles have a sieve diameter in the range from about 0.01 mm to about 10 mm, which means that they, or at least the majority of the particles, would normally pass through a sieve having an aperture or opening size of about 10 mm, but not through a sieve having an aperture or opening size of about 0.01 mm or less. Optionally, the particles may also have a diameter in the range from about 0.05 mm to about 3 mm, or from about 0.1 mm to about 2.5 mm, or from about 0.1 mm to about 2 mm, such as about 0.25±0.20 mm, about 0.5±0.25 mm, about 1.0±0.25 mm, about 1.5±0.25 mm, or about 2.0±0.25 mm, respectively. Within a composition comprising a plurality of particles according to the invention, these particle sizes should be interpreted to characterise the preferred mass median sieve diameters of the ingestible particles.

In some embodiments, the particles may even be larger, having a sieve diameter in the range from about 0.01 mm to about 20 mm, which the means that they, or at least the majority of the particles, would normally pass through a sieve having an aperture or opening size of about 20 mm, but not through a sieve having an aperture or opening size of about 0.01 mm or less. Typically, the particles with an approximately spherical or cuboidal shape (i.e. those having an aspect ratio of longest space diagonal divided by shortest space diagonal anywhere in the range of about 1 to 2.5), such as pellets, minitablets and certain granules, have a mass median sieve diameter in the range from 0.01 mm to 10 mm; whereas those particles with a more elongated or platelet like shape (i.e. aspect ratios larger than 2.5), such as flakes, shavings or chips, may in some cases exceed the 10 mm and have a mass median sieve diameter in the range from 0.01 mm up to 20 mm.

If the particles are to be swallowed as such, it is also preferred that they have a mass median sieve diameter in the range from about 0.1 mm to about 3 mm. Larger particles with a mass median sieve diameter in the range of up to 10 mm or even 20 mm on the other hand may easily be chewed, since the particles are not sticky when in contact with saliva. Also preferred are mass median sieve diameters in the range from about 0.5 mm to about 10 mm, or from about 0.6 mm to about 8 mm, or from about 0.65 mm to about 7 mm, or from about 0.7 mm to about 5 mm, or from about 0.75 mm to about 2.5 mm, or from about 1 mm to about 2 mm. In other preferred embodiments, the mass median sieve diameter may be in the range from about 0.1 mm to about 0.4 mm, from about 0.2 mm to about 0.5 mm, or from about 0.2 mm to about 0.4 mm, respectively.

The solid phase particles may be consumed as is or in combination with comestible goods, including ingestible liquids. For instance, flakes or chips of the solid phase may be blended into a muesli mixture or into ‘trail-mix’ compositions; pellets dispersed in milk or water; sprinkles added to yoghurt. Further details will be provided below.

Alternatively, the solid phase may be provided as such, without forming particles of it; the solid phase provided in the form of an ingestible non-particulate matrix. This matrix may be provided as is, e.g. shaped similar to a chocolate bar. However, considering the intended amounts of the solid phase to be administered as well as the taste, it is usually more preferred, to combine the matrix with solid comestible goods. For instance, the matrix may be applied as a layer on a wafer or a crispbread, or in between two or more wafer/crispbread layers; or it may be layered onto comestible goods like cornflakes, cereals, puffed rice, puffed soy or the like; or the matrix may be used like a binder, e.g. in muesli bars or protein bars. Further details will be provided below.

The water-soluble polysaccharide component is a hydrophilic or amphiphilic material based on glucose or fructose with an average degree of polymerisation from 2 to 100, or from 4 to 80. Preferably, the average degree of polymerisation is from about 5 to about 60. The water-soluble polysaccharide component is capable of dissolving in an aqueous environment. Preferably, the polysaccharide component exhibits a solubility of at least 2 wt.-%, or at least 3 wt.-%, or at least 4 wt.-% and optionally of at least 5 wt.-%, measured in purified water at 25° C.

In a preferred embodiment, the polysaccharide component comprises an essentially non-swelling and essentially non-mucoadhesive polymer; i.e. the polysaccharide component as such essentially does not have, or induce, any pronounced mucoadhesive properties. This provision applies equally to the water-soluble polysaccharide component as to the water-insoluble polysaccharide component.

As used herein, swelling by water, or in an aqueous environment, typically means the volume increase of a solid body caused by an influx, or diffusion process of water accompanied by hydration, i.e. wetting and absorption of moisture. Swelling may e.g. may expressed by the swelling value in percent calculated as (wₛ-w_{d})/w_{d}×100 (with w_{d}=initial weight of dry component and wₛ=weight of swollen component). For the purposes of this study, swelling, or swelling capacity, is to be understood as the swelling behavior in vivo and should thus be evaluated under conditions mimicking those in vivo; e.g. by placing a fixed amount (w_{d}) of the polysaccharide in excess drinking water of 37° C.±2° C. for 4 hours, before removing excess water with the help of a filter and weighing the weight of swollen component (wₛ). The term ‘non-swelling’ as used herein shall refer to a swelling value of not more than 10%, preferably not more than 5%.

Mucoadhesiveness, as use herein, is the capability of adhering to a mucosa, or mucosal membrane. Various conventional methods are available to determine mucoadhesiveness, such as tensile strength measurements, ellipsometry, or rheological measurements (D. Ivarsson et al., Colloids Surf B Biointerfaces, vol. 92, pages 353-359, 2012). Even though these methods may not provide absolute values for mucoadhesiveness as such, they indicate the presence and relative magnitude of mucoadhesiveness of a material.

To determine mucoadhesiveness in the context of the invention, it is preferred that a modified falling liquid film method (described among other methods in Mucoadhesive drug delivery systems, Carvalho F. C. et al., Brazilian Journal of Pharmaceutical Sciences 46 (2010)) is employed. According to the method, the selected mucous membrane (e.g. from pig stomach) is placed in a petri dish together with simulated gastric fluid at a controlled temperature of 37° C. The petri dish is placed on a table undergoing a tilting movement. Both tilting movement and volume of buffer are selected so that small waves of buffer continuously run over the surface of the mucous tissue. In the falling liquid film method, a similar agitation is achieved by pumping buffer over mucosal tissue tilted at a 45° angle. Particles or granules of the compound or material in question are placed on the tissue. The amount of particles remaining on the mucous membrane after a specified time interval can be quantified by various methods. For instance, particles can be counted, optionally using a magnifying glass or microscope, or they may be collected, dried and measured gravimetrically.

In a preferred embodiment, the solid phase of the invention comprises a neutral (i.e. non-ionic) polysaccharide that is resistant to digestion in the human small intestine. Examples of suitable neutral polysaccharides include, but are not limited to, glucans such as beta glucans; dextrins; fructans such as inulin; pullulans; cellulose or hemicellulose. Preferably, the neutral polysaccharide is a non-ionic dietary fibre, e.g. a soluble or an insoluble dietary fibre. Preferably, the neutral, water-soluble polysaccharide is selected from the group consisting of cereal-derived beta glucans such as oat beta glucan or barley beta glucan, inulins and resistant dextrins from starch. The neutral, water-insoluble polysaccharide may be selected, for instance, from the group consisting of cellulose, hemicellulose and long-chain and/or branched beta glucans such as bacteria derived curdlan.

Resistant dextrins are partially hydrolysed starches; i.e. short chain glucose polymers, without sweet taste which are water-soluble and relatively resistant to the hydrolytic action of human digestive enzymes. They can be made for instance from wheat (Nutriose® FB range or Benefiber®) or maize starch (Nutriose® FM range), using a highly controlled process of dextrinisation (heating the starch in the presence of small amounts of food-grade acid), followed by a chromatographic fractionation step. This produces a highly indigestible, water-soluble dextrin, with a high fibre content of about 65-85%, and a narrower, more favourable molecular weight distribution; e.g. approx. 4000 to 6000 Da for Nutriose® 6, or 3500 to 4500 Da for Nutriose® 10 (other dextrins, e.g. one of the starting materials to prepare resistant dextrins, may exhibit broader molecular ranges such as from about 3000 to 10,000 Da). During the dextrinisation step, the starch undergoes a degree of hydrolysis followed by repolymerisation that converts it into fibre and results in a drastically reduced molecular weight and the introduction of new glucoside linkages: in addition to the digestible α-1,4 and α-1,6 glycosidic linkages as commonly found in starches and the digestible maltodextrins, also non-digestible glycosidic bonds such as β-1,2 or β-1,3, are formed in resistant dextrins, which cannot be cleaved by enzymes in the digestive tract. As a result, a portion of the dextrin is not digested in the upper part of the gastro-intestinal tract and is not directly available as such for energy utilisation. Further, some commercial suppliers offer grades with different levels of mono- and di-saccharides (e.g. Nutriose® 10 Nutriose® 6, as available from Roquette), while the composition of the higher molecular weight oligomers is the same in both grades.

In one embodiment, the water-soluble polysaccharide component comprises a dextrin having a degree of polymerisation from 2 to 100, preferably from 4-40, or from 5 to 40, or from 8 to 40, or from 10 to 30 and further preferably from 12 to 25. In one embodiment, the water-soluble polysaccharide component comprises a dextrin having a dextrose equivalent of not higher than 20, preferably a dextrose equivalent of 5 to 15.

In another embodiment, the polysaccharide component is an inulin. Inulins are water-soluble, indigestible polysaccharides, naturally occurring as a storage carbohydrate in many types of plants such as chicory which is most often used for extraction of inulin. Inulins consist of glucose and fructose moieties which are linked by β-(2,1) bonds, which renders them indigestible to the enzymes in human GI-tract and contributes to the functional properties of inulins, such as reduced calorie value, dietary fibre, prebiotic effects and only very mild sweetening. The degree of polymerization (DP) of the inulin may range from 2 to 100. The degree of polymerization (DP) of standard inulin ranges from 2 to 60. In one embodiment, the polysaccharide component comprises an inulin having a degree of polymerisation from 4 to 60, or from 10 to 60, more preferably from 3 to 50, or from 4 to 30, or from 5 to 25.

In a further embodiment, the polysaccharide component is a fructooligosaccharide (FOS); also referred to as oligofructose or oligofructan. Similar to inulin, oligofructose is a naturally occurring storage carbohydrate in plants such as chicory and is based on 3-10 β-glycosidically linked fructose units.

Inulin and oligofructose are commercially available from Raffinerie Tirlemontoise S. A., Brussel as Raftilin® or Raftilose®. Further fructooligosaccharides are commercially available; e.g. Actilight® from Cerestar/Cargill.

In a further embodiment, the water-soluble polysaccharide component is a soluble glucan. Non-limiting exemplary glucans include polydextrose, cyclodextrin, soluble beta-glucans and mixtures thereof. In one advantageous embodiment, the glucan is polydextrose. Polydextrose (also known as E1200) is a water-soluble fibre synthesized from dextrose (glucose), about 10% sorbitol and 1% citric acid; commercially available e.g. as Litesse® from Danisco. In a further advantageous embodiment, the soluble glucan is a soluble beta-glucan. Beta-glucans (β-glucans) are polysaccharides of D-glucose monomers linked by β-glycosidic bonds, which occur e.g. in the bran of cereal grains. Some forms of beta-glucans such as those derived from oats or other cereals are useful as soluble dietary fibres, since oat is rich in the water-soluble yet indigestible fibre (1,3/1,4) β-glucan. In addition, oat beta glucan (e.g. commercially available as PromOat® from Tate&Lyle) has been shown in clinical trials to lower blood cholesterol and reduce post-prandial glycaemic response. In a yet further embodiment, the glucan is a cyclodextrin. Cyclodextrins are commercially available from Wacker (Germany); for instance Cavamax®, alpha-cyclodextrin, is available in food-grade qualities.

As mentioned, the water-soluble polysaccharide component in the solid phase described herein may be replaced by a water-insoluble, non-swelling, edible polysaccharide component. Examples of suitable water-insoluble, non-swelling, edible polysaccharide component include cellulose, hemicellulose or long-chain and/or branched beta-glucans like curdlan.

Curdlan is an example of a neutral, high-molecular-weight, essentially linear and non-digestible beta-(1,3)D-glucan approved for usage in food products. Unlike the water-soluble cereal derived beta-glucans, curdlan is produced by bacteria such as Alcaligenes faecalis and water-insoluble. At body temperature, curdlan forms suspensions; only upon heating the suspensions to temperatures above 80° C., elastic gels are formed.

Optionally, the polysaccharide component according to the invention comprises more than one polysaccharide component as defined herein.

In a preferred embodiment, the solid phase of the invention comprises not more than 5 wt.-% of mucoadhesive polymer. Optionally, the content of the mucoadhesive polymer in the solid phase is not more than about 4 wt.-%, or not more than 3 wt.-%, or not more than 2 wt.-%, or not more than 1 wt.-%, respectively. In one embodiment, the solid phase is free or substantially free of mucoadhesive polymer. In yet another embodiment, the solid phase essentially consists of the water-soluble polysaccharide component and the fatty acid glyceride component as defined herein, and optionally one or more further excipients without mucoadhesive properties, such as sugars, sugar alcohols, vitamins, amino acids, proteins and/or micro-nutrients.

However, if a minor amount (as described above) of a mucoadhesive material is incorporated into the solid phase of the invention, such material may be selected from:

(a) poly(carboxylates) such as poly(acrylic acid) (optionally in crosslinked form, e.g. carbomers like Carbopol® or polycarbophils like Noveon® AA-1), poly(methacrylic acid), copolymers of acrylic and methacrylic acid, and poly(hydroxyethyl methacrylic acid), alginic acid or salts thereof, or pectins;
(b) cellulose ethers such as carboxymethylcellulose hydroxyethyl cellulose, hydroxypropyl cellulose (also known as hyprolose), hydroxypropyl methylcellulose (also known as hypromellose), and methylcellulose,
(c) mucoadhesive polysaccharides like chitosan, gellan gum, guar gum, or xanthan gum, and
(d) gum arabic (a mixture of glycoproteins and polysaccharides).

According to the present invention, the fatty acid glyceride component has a melting point or melting range of higher than 37° C. In the context of the present invention, the melting point refers to the fatty acid glyceride component as such, i.e. not in its hydrated state, and it should be understood as the temperature at which the glyceride component melts entirely, without solid residue, at normal pressure. If a glyceride component exhibits a broad melting range, the melting point is understood as the higher limit of the range

In one embodiment, the fatty acid glyceride component has a melting point from 38° C. to 75° C., in particular from 40° C. to 70° C., or from 40° C. to 65° C. In blends, at least the fatty acid glyceride component and/or at least one of the fatty acid glyceride components has a melting point or melting range of higher than 37° C. In a preferred embodiment, the mass fraction of low-melting constituents (mp below 37° C.) in the fatty acid glyceride component is limited to less than 20%, preferably less than 10%, and more preferably less than 5% or even less than 2%. In case the content of low-melting fractions is higher, the fatty acid glyceride component may potentially be perceived as sticky, or “gluey”, upon mastication.

The fatty acid glyceride component is preferably a mono-, di- or triglyceride, and comprises at least one medium or long chain fatty acid residue. In the case of di- and triglycerides, these may have different fatty acid residues per glyceride molecule. The acyl chain of the fatty acid may be saturated or unsaturated.

Optionally, fatty acid glycerides components having a high content of triglycerides are used, e.g. with at least 60 wt.-% triglycerides, or at least 70 wt.-% triglycerides, or at least 80 wt.-% triglycerides, or at least 90 wt.-% triglycerides. Optionally, the glyceride substantially consists of triglyceride(s). Moreover, it is also preferred that the content of the fatty acid triglyceride in the particle is at least 10 wt.-%. Optionally, the content of the fatty acid triglyceride in the particle is at least 20 wt.-%, or at least 30 wt.-%, or at least 40 wt.-%, or at least 50 wt.-%, respectively.

The constituent(s) of the fatty acid glyceride component may represent a native, synthetic or semisynthetic material. In a preferred embodiment, the fatty acid glyceride component is a native material, i.e. obtained from a natural source by extraction, but without chemical modification. For example, a native or natural triglyceride is not hardened or hydrogenated. Irrespective of the source of the constituent(s) of the fatty acid glyceride component, non-hydrogenated constituents are preferred over partially hydrogenated or fully hydrogenated constituents; and partially hydrogenated are again preferred over fully hydrogenated ones due to e.g. the occurrence of trans-fatty acids. Furthermore, “non-trans-esterified” constituents are preferred over “trans-esterified” ones.

A medium chain fatty acid is understood as fatty acid with an acyl residue of 6 to 12 carbon atoms, whereas a long chain fatty acid means a fatty acid with an acyl chain of 13 to 21 carbon atoms. Among the preferred medium chain fatty acids are capric acid and lauric acid, including their esters and salts, in particular their mono-, di- and triglycerides and their sodium, potassium and calcium salts. Examples of preferred long chain fatty acids include myristic acid, palmitic acid, stearic acid, arachidic acid, behenic acid, myristoleic acid, palmitoleic acid, sapienic acid, oleic acid, linoleic acid, and linolenic acid, and the respective salts and glycerides. While the incorporation of polyunsaturated fatty acids such as linoleic acid to the ingestible particles offers a number of health benefits, this should be done with care in order to not render the fatty acid glyceride component sticky or “gluey”; preferably their mass content in the ingestible particles should not surpass 10 wt.-%.

In another embodiment, the fatty acid glyceride component comprises one or more partial glycerides of a medium or long chain fatty acid, in particular monoglycerides of a medium or long chain fatty acid. For example, monolaurin would be suitable for carrying out the invention. As used herein, a monoglyceride such as monolaurin may be incorporated as a substantially pure compound or as a mixture of mono- and diglycerides or even mono-, di- and triglycerides with various fatty acids, but with a high content (“enriched”) of a particular monoglyceride compound. For example, a monolaurin grade may be used which comprises at least about 40% (or at least about 50%, or 60% or 70% or 80% or 90%) of the actual monoglyceride of lauric acid.

A preferred fatty acid glyceride component having a melting point or melting range of above 37° C. is fractionated but non-hydrogenated palm stearin or palm kernel stearin. Palm stearin is the solid fraction of palm oil that is produced by partial crystallization at controlled temperature. It is commercially available e.g. as Prifex® 300 from Sime Darby Unimills.

In one specific embodiment, the solid phase may comprise a water-soluble polysaccharide component comprising a resistant dextrin derived from wheat or maize starch (such as Nutriose® or Benefiber® or the like) and a fatty acid glyceride component comprising a fractionated but non-hydrogenated palm stearin or palm kernel stearin (such as Prifex® or the like).

It was surprisingly found by the inventors, that the solid phases of the invention—and in particular solid phases comprising mixtures of resistant dextrins and a fractionated but non-hydrogenated palm stearin or palm kernel stearin as the fatty acid glyceride component—exhibit a surprisingly pleasant mouthfeel.

The term ‘mouthfeel’ as used herein covers on the one hand texture properties as defined by tactile perceptions of pressure and contact in the mouth (e.g. fatty, creamy, oily, viscous, sticky, liquid, powdery, sandy, grainy). On the other hand, kinaesthetic perceptions during biting, chewing and swallowing are included (e.g. crunchy, firm, brittle, crumbly, crispy, resilient). Furthermore, geometric properties of a food (e.g. size, form and amount of broken pieces), properties connected with the water content of the food (juicy, dry) as perceivable by tongue and palate are involved in the mouthfeel too; as well as all properties based on temperature perception (hot, cooling) and irritating/painful perceptions of free nerve endings (e.g. spicy, tingling, burning).Commonly, high melting a fatty acid glyceride components do not provide a good mouthfeel in that they cannot melt or noticeably soften at body temperature and are thus mostly perceived as too hard and/or too ‘plastic-like’, like ingesting a foreign object rather than something edible. The addition of the polysaccharide component as defined herein- and in particular the addition of the resistant dextrins—to the solid phase helps to improve the mouthfeel, e.g. by making the fatty acid glyceride component appear softer and/or smoother, yet crunchy, or crispy, at the same time. Chewability is also improved in that the solid phase appears less ‘plastic-like’ or brittle. Advantageously, the solid phase does not get sticky or slimy by the addition of the polysaccharide components such as the resistant dextrins, so that it can be chewed, or masticated, easily without e.g. adhering to the gums and/or teeth.

Without wishing to be bound by theory, it is suggested that by adding the polysaccharide component, such as the resistant dextrin, to the solid phase, water can be absorbed and/or adsorbed more easily, thereby rendering the mouthfeel of the hard, high-melting fatty acid component softer, less dry, ‘plastic-like’ or brittle and overall more ‘food-like’. In addition, absorbed water may potentially reduce the melting temperature of the fatty acid component just enough to allow for partial melting to occur at the surface layer of the ingestible particles (or masticated pieces) which is likely perceived by the tongue as being more pleasant.

In one of the preferred embodiments, the water-soluble polysaccharide component and of the fatty acid glyceride component make up the majority of the mass of the solid phase so that the combined content of the water-soluble polysaccharide component and of the fatty acid glyceride component in the solid phase is at least about 50 wt.-%. Optionally, the combined content of the water-soluble polysaccharide component and of the fatty acid glyceride component in the solid phase is at least 60 wt.-%, or at least 70 wt.-%, or at least 80 wt.-%, or at least 90 wt.-%, or at least 95 wt.-%, respectively. The solid phase may also substantially consist of the water-soluble polysaccharide component and the fatty acid glyceride component. Same applies for the solid phase comprising a water-insoluble, non-swelling, edible polysaccharide.

In one embodiment, the solid phase further comprises one or more free fatty acids. For example free oleic acid or lauric acid may be part of the lipid component. Other preferred free fatty acids are mixtures of unsaturated fatty acids such as the so-called omega fatty acids or conjugated linoleic acids (CLA), a family of isomers of linoleic acid. Brands of CLAs are marketed as dietary supplements (Tonalin®, BASF, and Clarinol®, Stepan). Examples of omega-3 fatty acids are α-linolenic acid (ALA) (found in plant oils), eicosapentaenoic acid (EPA), and docosahexaenoic acid (DHA) (both commonly found in marine oils). If the fatty acid glyceride component comprises an unsaturated fatty acid, the particle according to the invention may optionally comprise an antioxidant such as vitamin E or a derivative thereof.

It has been surprisingly found by the inventors that solid phase particles containing the polysaccharide component as defined herein in intimate mixture with a fatty acid glyceride component as defined herein are capable of exhibiting a prolonged integrity of the particles; same applies to the masticated pieces of the solid phase in general. Possibly, this contributes to, or is related to, the increased bioavailability of the fatty acid glyceride component(s)—as for instance measured by increased cPDR in a breath test study (see Example 2)—and the improved induction of satiety caused by the particles' administration. Without wishing to be bound by theory, this increase in bioavailability may e.g. be caused by the prolonged integrity of the particle(s) according to the invention which may result in more rapid gastric emptying of the particle(s).

Typically, the preparation of the intimate mixture means that the fatty acid glyceride component and the polysaccharide component are mixed in such a way that the porosity of the resulting fatty acid glyceride-polysaccharide composition is greatly reduced as compared to the particles formed from the polysaccharide component itself, for instance as formed by roller compaction or agglomeration. This probably contributes to, or is related to, the increased solid phase particles as described above. Particle porosity may be determined by porosimetry, an analytical technique used to determine various quantifiable aspects of a material's porous nature, such as pore diameter, total pore volume, and surface area. The technique involves the intrusion of a non-wetting liquid at high pressure into a material through the use of a porosimeter.

As already discussed, it is a key feature of the invention that the polysaccharide component is in an intimate mixture with the fatty acid glyceride component, which appears to effect an improved and/or prolonged interaction of the fatty acid glyceride component with their target at the gastrointestinal mucosa. A target structure may, for example, be represented by G-protein coupled receptors (GPCRs) involved in the sensing of intestinal lipids such as GPR120.

In some embodiments, this may also result in an increased bioavailability of the fatty acid glyceride component. In this context, bioavailability should be broadly understood such as to include the availability of e.g. the fatty acid glyceride component, or the biologically active constituents thereof, at a biological target site, such as the gastric or intestinal mucosa, in terms of the extent and/or duration of availability.

To further enhance the beneficial effects of the solid phase, it is preferred that the weight ratio of the fatty acid glyceride component to the polysaccharide component is in the range from about 0.1 to about 10. In some embodiments, the weight ratio is from about 0.1 to about 5, from about 0.3 to about 3, from about 0.5 to about 2, respectively. Preferred is a weight ratio from about 0.5 to about 5. For the avoidance of doubt, these preferred ratios are intended as a general teaching and are applicable to all alternative embodiments of the composition of the invention with respect to the selection of components A, B, C, D and/or E, and apply to all uses of the compositions.

The inventors have found that the satiety-inducing effect of a free or esterified fatty acid is enhanced if delivered in the form of the solid phase of the invention, which allows appetite suppression and the prevention and/or treatment of obesity even without pharmacological intervention using a synthetic drug. It is therefore a preferred embodiment that the solid phase is free of a synthetic drug substance. In other words, the solid phase may substantially consist of the polysaccharide component and the fatty acid glyceride component, and optionally the components described above and/or other pharmacologically inert excipient(s) such as an inert core.

Optionally, the solid phase may further contain an amino acid, a protein, a vitamin, a micro-nutrient, or any combinations of these.

As used herein, an amino acid is an organic compound having an amino group and a carboxyl group, mostly in the generic structure of NH₂-CHR-COOH wherein R represents the side chain which is specific to each amino acid. Optionally, the carboxylic group is partially or fully neutralised. The amino acid may be provided in its L-form, its D-form or in its racemic form. In a preferred embodiment, the amino acid is a proteogenic amino acid, i.e. an amino acid which is a potential precursor of a protein in that it may be incorporated into a protein during its translation, or biosynthesis. Proteogenic L-amino acids as currently identified are L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, L-valine, L-selenocysteine, L-pyrrolysine, and N-formyl-L-methionine. In another embodiment, the amino acid is selected from the 20 amino acids which form the genetic code, which group consists of L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine.

In another embodiment, the amino acid is selected from the group of the so-called essential amino acids which consists of those amino acids which the human organism cannot synthesise, i.e. L-histidine, L-isoleucine, L-leucine, L-lysine, L-methionine, L-phenylalanine, L-threonine, L-tryptophan, and L-valine.

In a further embodiment, the amino acid is selected from the group consisting of L-isoleucine, L-valine, L-tyrosine, L-methionine, L-lysine, L-arginine, L-cysteine, L-phenylalanine, L-glutamate, L-glutamine, L-leucine, and L-tryptophan. From these, the group consisting of L-phenylalanine, L-leucine, L-glutamine, L-glutamate, and L-tryptophan is particularly preferred. In another preferred embodiment, the amino acid is L-tryptophan.

Optionally, the solid phase comprises two or more amino acids. Such mixture or combination of amino acids should preferably comprise at least one amino acid as described above, i.e. a proteogenic amino acid, or an amino acid from the group of amino acids forming the genetic code, or from the essential amino acids, or the group of amino acids consisting of L-isoleucine, L-valine, L-tyrosine, L-methionine, L-lysine, L-arginine, L-cysteine, L-phenylalanine, L-glutamate, L-glutamine, L-leucine, and L-tryptophan. Particularly preferred solid phases with mixtures or combinations of amino acids comprise at least one amino acid from the group consisting of L-phenylalanine, L-leucine, L-glutamine, L-glutamate, and L-tryptophan. In particular, L-tryptophan is a preferred constituent of a combination of two or more amino acids.

Also preferred are mixtures or combinations of amino acids in which at least two amino acids are members of one of the preferred groups as previously defined. Moreover, mixtures or combinations of amino acids may be used in the solid phase of the invention in which essentially all incorporated amino acids are members of one of the preferred groups as previously defined.

As used herein, proteins are macromolecules, consisting of one or more chains of amino acid residues, typically characterized by a specific three-dimensional structure determining the protein's activity, the so-called protein folding. The protein can be native or modified and can be derived from vegetable or animal sources. The term ‘proteins’ as used herein also includes longer linear chains of amino acid residues with typically more than 20-30 amino acid residues; so-called polypeptides. If present, the ingestible particles preferably comprise one or more proteins selected from the group of legume proteins, grain proteins, nut proteins, mushroom proteins, and proteins from the seeds of other plants, milk proteins, egg proteins and gelatin. Particularly suitable vegetable proteins include soy protein, rice protein, hemp seed protein, pea protein lupin protein and almond protein.

As used herein, vitamins are vital nutrients required in small amounts, which e.g. humans (or other organisms) typically cannot synthesise in sufficient quantities and which therefore must be taken up with the diet. The term ‘vitamin’ is conditional in that it depends on the particular organism; for instance ascorbic acid is a vitamin for humans, while many other animals can synthesise it. Vitamins are organic compounds classified by their biological and chemical activity, not by their structure. Each vitamin refers to a number of vitamers, all having the biological activity of the particular vitamin, convertible to the active form of the vitamin in the body, and grouped together under alphabetised generic descriptors, such as ‘vitamin A’. Universally recognised vitamins are preferred for the present invention (related vitamer(s) in brackets): vitamin A (retinol, retinal, and the carotenoids, including beta carotene, cryptoxanthin, lutein, lycopene, zeaxanthin), vitamin B1 (thiamine), vitamin B2 (riboflavin), vitamin B3 (niacin, niacinamide), vitamin B5 (pantothenic acid), vitamin B6 (pyridoxine, pyridoxamine, pyridoxal), vitamin B7 (biotin), vitamin B8 (ergadenylic acid), vitamin B9 (folic acid, folinic acid), vitamin B12 (cyanocobalamin, hydroxycobalamin, methylcobalamin), vitamin C (ascorbic acid), vitamin D (cholecalciferol (D3), ergocalciferol (D2)), vitamin E (tocopherols, tocotrienols), vitamin K (phylloquinone, menaquinones). The vitamins according to the invention may be provided as semisynthetic and synthetic-source supplements and/or as supplements of natural origin; such as in the form of plant extracts.

As used herein, the term ‘micro-nutrients’ refers to nutrients required by humans and/or other organisms in small quantities for a variety of their physiological functions, their proper growth and development; including, for instance, dietary micro-minerals or trace elements in amounts generally less than 100 mg/day (as opposed to macro-minerals). The micro-minerals or trace elements include at least boron, bromine, cobalt, chromium, copper, fluoride, iodine, iron, manganese, molybdenum, selenium, and zinc. They may optionally be present in ionised or complexed form or as a salt, an oxide or a chelated salt. Micro-nutrients also include phytochemicals, such as terpenoids or polyphenolic compounds, organic acids, choline, cholesterol as well as vitamins (i.e. some compounds may qualify for both categories, vitamins and micro-nutrients).

Preferred micro-nutrients according to the invention may be selected from organic acids, such as acetic acid, citric acid, lactic acid, malic acid, and taurine; and trace -or micro-minerals such as salts of boron, bromine, cobalt, chromium, copper, fluoride, iodine, iron, manganese, molybdenum, selenium, or zinc; choline and cholesterol.

These optional components, e.g. the amino acid, the protein, the vitamin and/or the micro-nutrient may be incorporated within the solid phase of the invention in different ways. For example, hydrophilic compounds such as amino acids, water-soluble vitamins and water-soluble micro-nutrients may be incorporated in admixture with the water-soluble polysaccharide component, whereas lipophilic compounds may be incorporated in admixture with the fatty acid glyceride component.

The particle according to the invention may be in the form of a granule, a pellet, a minitablet, a flake, a shaving, a chip, a sprinkle or mixtures thereof. More preferably, the particle is a granule, a pellet and/or a flake. However, it should be noted, that the satiety inducing effect of the solid phase of the invention usually does not rely on the specific shape of the solid phase particle but on the solid phase's composition.

As used herein, a granule refers to an agglomerated particle which has been prepared from a plurality of smaller, primary particles. Hence, as used herein the term granule(s) does not necessarily imply a specific shape, since the final shape of the granule(s) will be guided by the specific method of preparation. Agglomeration, or granulation, for the purpose of preparing a granule, may involve the use of a dry, wet or melt granulation technique as will be detailed further below.

A pellet, as used herein, is understood as a particle with a relatively spherical or spheroidal shape. If prepared by an agglomeration process, a pellet is a special type of a granule. However, pellets (i.e. spherical or spheroidal particles) may also be prepared by other processes than agglomeration. For the avoidance of doubt, the degree of sphericity of a pellet may differ in various technical fields. In the context of the invention, the sphericity of a pellet is in the typical range of pellets used in pharmaceutical formulations for oral use, which often have an aspect ratio of longest space diagonal divided by shortest space diagonal in the range of about 1 to 1.5.

A minitablet, often also referred to as a microtablet, is a unit formed by the compression or compaction of a powder or of granules. Typically, the compression is done on tablet presses using punches.

Minitablets, tablets or capsules comprising the particles of the invention are preferably formulated and processed in such a way that they rapidly disintegrate after oral administration. As used herein, disintegration is understood as a substantial physical change to the minitablet, tablet or capsule morphology, such as the rupture or detachment of the tablet's coating, the dissolution of a capsule or the disintegration of a tablet or minitablet to release particles or pellets or granules of the invention. For the detection of such tablet, minitablet or capsule disintegration behaviour, a microscope may be used. With respect to the apparatus, the hydrodynamic conditions, and the temperature, the method <701> of the United States Pharmacopeia 29 (USP29) may be used, except that water may be used as test medium and that the wire mesh may be adapted with respect to the mesh size or aperture to take the sieve diameter of the tablet, minitablet or capsule into account. When tested according to this method, the minitablets or tablets or capsules comprising particles according to the invention preferably disintegrate within not more than about 15 minutes. More preferably, they disintegrate within about 10 minutes or less. According to another embodiment, they disintegrate within about 8 minutes or less, or within about 5 minutes or less, respectively.

A flake as used herein is understood a flat, approximately platelet-like shaped piece of solid material, namely the solid phase of the invention, commonly exhibiting an aspect ratio of longest space diagonal divided by shortest space diagonal larger than 2.5. For instance the aspect ratio may be 3, 4, 5 or larger. In cases where the preparation process involves a casting step (as will be detailed below), the flakes are commonly rectangular in cross-sectional view and exhibit an approximately uniform thickness, while in top view the flakes may have various shapes ranging from triangular to polygonal, e.g. rectangular. The shape in top view depends mainly on how the larger cast material layer is transferred into particles/flakes, e.g. whether it is broken or cut. Breaking commonly leads to more irregular shapes, such as rectangles broken in halves or with chipped corners. Alternatively, the solid phase may even be cast into moulds which define the final shape of the flake(s) already, such that the cast material does not require breaking or cutting anymore. In one embodiment, the flakes may e.g. be rectangles or squares with a side length of 2 mm to 20 mm and a thickness of about 0.01 mm to about 3 mm.

Shavings as used herein refers to thin, irregular shaped pieces of solid material, namely the solid phase of the invention, which are obtained from a larger block of the material by scraping, grating or shaving, e.g. using a wire, blade, or plough. Shavings are mostly thinner than the flakes as defined above, and thus may fold, roll or curve when removing them from the larger block. In an optional embodiment, the consumer may be provided with the larger block of material in order to freshly prepare the shavings prior to use.

Chips as used herein are similar to the flakes as defined above, in that they are pieces of solid material, namely the solid phase of the invention, commonly exhibiting an aspect ratio of longest space diagonal divided by shortest space diagonal larger than 2.5. For instance the aspect ratio of the chips may be 3, 4, 5 or larger. Chips of the solid material may e.g. be prepared by casting defined portions of the solid phase in softened or semi-solid form from a nozzle onto a surface, and allowing said portion to solidify on said surface. This process leads to the typical shape of the chips, which are spherical or spheroidal in top view and curved, or half-elliptical or half-spherical, in cross-sectional view. Examples of such shapes may be found in the confectionery field where chocolate raw masses are available as so-called Callets®. Also various suppliers of solid lipids deliver their raw materials in this chip(s) form.

Sprinkles as used herein are small cylindrical, or rod-shaped pieces of solid material, namely the solid phase of the invention, commonly exhibiting an aspect ratio of longest space diagonal divided by shortest space diagonal larger than 2.5. For instance the aspect ratio of the chips may be 3, 4, 5 or larger, such as cylinders with a diameter of about 0.01 mm to about 3 mm and a length of about 4 mm to about 10 mm. Their shape is similar or equal to the shape of e.g. sugary sprinkles as known from the confectionery field.

The solid phase according to the invention may be prepared by a method comprising the steps of
(i) preparing an intimate mixture comprising the polysaccharide component and the fatty acid glyceride component (for instance, by melting the fatty acid glyceride component at least partially and blending in the water-soluble polysaccharide component), and
(ii) applying the intimate mixture onto comestible goods prior to solidification;
(iii) blending the intimate mixture with comestible goods prior to solidification; or
(iv) processing the intimate mixture further to obtain ingestible particles of said mixture by (a) extruding the mixture using a screw extruder; (b) spray congealing the mixture, optionally using a jet-break-up technique; (c) melt granulating the mixture; (d) compressing the mixture into minitablets; (e) melt injection of the mixture into a liquid medium; or (f) spray coating of the mixture onto inert cores. Alternatively, the water-soluble polysaccharide component may be replaced in this preparation method by a water-insoluble, non-swelling, edible polysaccharide component.

The preparation of the intimate mixture comprising the polysaccharide component and the fatty acid glyceride component may be accomplished by conventional means such as blending or high-shear mixing.

Optionally, the intimate mixture is prepared using the same equipment which is also utilised for the subsequent step in which the particles are formed. For example, for preparing a melt to be used for melt congealing, melt granulation or melt injection, it may not be required to first prepare a dry premix prior to melting the constituents of the melt, but the mixing and melting (or at least partial melting) can be performed simultaneously in one step. Therefore, the mixture to be processed according to steps (a) to (f) above should be broadly interpreted to cover any form of combining the materials required for preparing the particles.

In one embodiment, the preparation and/or the processing of the intimate mixture may involve a step of melting the fatty acid glyceride component at least partially and blending in the polysaccharide component.

The wording ‘melting at least partially’ as used herein means that the melting step does not necessarily have to be performed at temperatures high enough and/or processing times long enough to turn the fatty acid glyceride component into a liquid; a ‘partial’ melt in the form of a semi-solid, pliable mass may be suitable and in some embodiments even preferred for the invention in terms of e.g. mixing efficiency and/or energy consumption.

It should further be understood, that the step of melting the fatty acid component and the step of blending in the polysaccharide component may occur simultaneously (e.g. a powder blend of the two components being stirred while gradually heated) or in succession (i.e. preparing the melt or partial melt first and only then blending in the polysaccharide component).

The intimate mixture of the fatty acid glyceride component and the polysaccharide component may then be used as such, i.e. without forming particles from it. For instance in one embodiment, the intimate mixture may be applied, or layered, onto comestible goods prior to solidification; e.g. onto flat layers such as wafers, crispbread, softbread, or the like. Optionally, two or more layers of wafers, crispbread etc. may be combined, with the intimate mixture in between such that the mixture acts like a binder. Alternatively, the intimate mixture may be applied, or layered, as a matrix-layer onto smaller comestible goods particles such as ‘puffed’ goods like rice puffs, soy puffs, peanut puffs, or the like. The coated ‘puffs’ (also called ‘nuggets’) can be either consumed as such or mixed e.g. into yoghurt.

In another embodiment, the intimate mixture may be blended with comestible goods prior to solidification, e.g. with cereals, corn flakes, dried fruits or vegetables, seeds, nuts, protein powders or granules, spices, sugar, sugar alcohols, or the like. In this embodiment, the intimate mixture may be used as a binder similar to e.g. using honey, rice syrup or corn syrup in sweet muesli bars. Since the solid phase of the invention is taste-neutral (i.e. neither exhibiting a distinct sweet, salty, sour, bitter nor umami taste sensation), it is suited as a binder for both savoury as well as sweet snack bars and/or ‘crunchier’.

Furthermore, these compositions comprising the comestible goods and the solid phase of the invention may be shaped and packaged in the form of snack-bars, or muesli-bars or bite-sized portions; optionally packaged for single use/consumption.

In an alternative embodiment, the intimate mixture is processed further to obtain ingestible particles of said mixture. As mentioned above, the mixture may be (a) extruded using a screw extruder; (b) spray congealed, optionally using a jet-break-up technique; (c) melt granulated; (d) compressed into minitablets; (e) melt injected into a liquid medium; or (f) spray coated onto inert cores.

In one embodiment, the mixture is extruded using a screw extruder. Optionally, a twin-screw extruder is used for carrying out the extrusion step. The extruder should have a screen with an aperture that is useful for producing an extrudate with appropriate diameter, such as 0.5 mm or 1.0 mm as commonly used for the preparation of pellets. Of course, larger aperture diameters are feasible, too; for instance when aiming for larger, ‘coin’ shaped flakes which may be cut from extruded strands of e.g. 10 mm diameter. The screw speed may be selected in consideration of the capability of the extruder and on the processability of the mixture. For example, it may be useful to select a screw speed in the range from about 20 rpm to about 100 rpm.

Preferably, the extrusion step is carried out without the use of a solvent and at a relatively low temperature, such as below about 45° C., or below 40° C., or below 35° C., or below about 30° C., e.g. at room temperature. It is also preferred that the extrusion step is carried out at a temperature which is lower than the melting point of the fatty acid glyceride component, e.g. 20° C. below the melting temperature. This prevents leakage from the extruder as well as improving the mixing efficiency.

In one embodiment, the ingredients used for preparing the particles by extrusion are mixed or blended before they are fed to the extruder. Alternatively, the ingredients may be mixed using the same equipment which is utilised for the extrusion step. Thus, it is also preferred that the ingredients used for preparing extruded particles are provided to the extruder by co-feeding, using appropriate feeding equipment, and optionally recycled within the extruder (e.g. via internal bypass-loops) until a uniform, intimate mixture is obtained which is ready for subsequent extrusion.

Subsequent to the extrusion step, the extrudate may be spheronised in order to obtain approximately spherical particles. For this purpose, any conventional spheroniser may be used. The temperature of the spheroniser jacket should preferably be set to be lower than the lower limit of the melting range of the lowest-melting constituent of the mixture. The speed of the spheronisation plates may be set between about 200 rpm and about 2,000 rpm, such as about 500 rpm to about 1,500 rpm. Subsequent sieving may be performed in order to select an optimal particle size of the product.

Alternatively, the extrudate strand, e.g. a cylindrical strand with a diameter of 0.5 mm or 1 mm, may be cut into pieces of about 4 mm to about 10 mm length and left to solidify without spheronisation, thereby forming sprinkles.

In a particular embodiment, the particles are prepared from the mixture by spray congealing. This process may also be referred to as spray chilling or spray cooling. In this process, a liquid melt is atomised into a spray of fine droplets of approximately spherical shape inside a spray cooling chamber. Here, the droplets meet a stream of air or gas which is sufficiently cold to solidify the droplets. The air or gas stream may have a co-current, mix-current or counter-current direction of flow.

To improve the formation of droplets of appropriate size and shape, a heatable rotary spray nozzle or a fountain nozzle may be used. In the context of the invention, a high speed rotary nozzle is one of the preferred nozzle types for preparing the particles.

Optionally, the uniformity of the atomised droplets may be further enhanced by using a jet break-up technique, such as electrostatic droplet generation, jet-cutting, jet excitation or flow focusing. In general, jet break-up refers to the disintegration of a liquid/gas jet due to forces acting on the jet.

In electrostatic droplet formation processes, a nozzle equipped with an electrode is used which applies an electrical charge to the melt spray. In jet cutting, the spray is directed through a cutter similar to e.g. a rotary disc with apertures of defined size. Jet excitation means the excitation of the melt spray by ultrasonic waves, causing vibration and facilitating the separation of droplets.

Flow focusing results from combining hydrodynamic forces with a specific geometry, which may be achieved by using a pressure chamber pressurised with a continuous focusing fluid supply. Inside, a focused fluid is injected through a capillary feed tube whose extremity opens up in front of a small orifice linking the chamber with the exterior ambient. The focusing fluid stream moulds the fluid meniscus into a cusp giving rise to a microjet exiting the chamber through the orifice. Capillary instability breaks up the stationary jet into homogeneous droplets.

In another specific embodiment, the particles are prepared by injecting the melted mixture into a liquid. The liquid may be cooled to a temperature below room temperature, or preferably to substantially below the lower limit of the melting range of the lowest-melting constituent of the lipid component. The liquid should be selected taking the composition of the mixture into consideration, but also with an eye on safety and physiological tolerability. In many cases, ethanol is a suitable liquid.

In another embodiment, the particles may be formed by melt agglomeration, or melt granulation. In the context of the invention, agglomeration and granulation may be used interchangeably. For this purpose, the constituents of the mixture are mixed or blended and agglomerated, or granulated, in a suitable type of equipment, such as a heatable granulator, a high-shear mixer/granulator or a fluid bed granulator. Depending on the type of equipment, the granulation may be carried out by heating the mixture to a temperature at which at least one of its constituents softens or melts, under continuous stirring or mixing. In a conventional granulator, this may lead to larger agglomerates which are then passed through a sieve to obtain the desired particle size. If fluid bed equipment is used, the complete mixture may be fluidised and heated carefully up to the melting temperature of the lowest-melting constituent. Alternatively, the lowest-melting constituent may be melted and sprayed onto the fluidised powder mixture comprising the remaining constituents.

Optionally, the melt granules may be further processed and compressed into minitablets. For this purpose, it is preferred that the granules are first blended with one or more tablet fillers/binders to enhance the plasticity of the mixture. Moreover, conventional excipients to improve the flow of the granules and reduce their tackiness may also be added before compression. Tableting may be carried out using any conventional pharmaceutical tablet press, such as an eccentric press or a rotary press. Optionally, the press may be equipped with multi-punch tooling so that each compression yields a plurality of minitablets. Punches for very small tablet diameters are preferred for particles intended to be swallowed as such, such as between about 1 mm and about 3 mm, such as about 1.5 mm. For larger particles which are intended to be chewed, larger tablet diameters may be used, such as in the range from about 1 mm to about 10 mm.

Alternatively and depending the mixture's flow properties, the mixture of fatty acid glyceride component and polysaccharide component may also be compressed into minitablets as such; i.e. without a preceding melt granulation step.

In a further embodiment, the particles are prepared by spray coating the mixture comprising the fatty acid glyceride component and the polysaccharide component onto inert cores. As used herein, an inert core is a particle from a physiologically acceptable material which itself does not substantially contribute to the physiological effect of the particles of the invention, i.e. the induction of satiety. Examples of suitable cores include crystals of appropriate size and shape, such as sugar (sucrose) crystals. In one of the preferred embodiments, spherical beads or non-pareils made from sugar, starch, cellulose, in particular microcrystalline cellulose (e.g. Cellets®) are spray coated with the mixture.

The spray coating of the inert cores may, for example, be performed in a fluid bed apparatus. The mixture of the fatty acid glyceride component and the polysaccharide component may be melted and sprayed onto the fluidised core particles. Any optional components such as amino acid(s), protein(s), vitamin(s), micro-nutrient(s) or the like may also be added to this mixture. Alternatively, an aqueous or organic dispersion (or suspension, which is understood as a sub-type of a dispersion) of the mixture is sprayed onto the fluidised cores in such a way that the water or solvent evaporates and the mixture of the fatty acid glyceride component and the polysaccharide component forms a coating on the inert core particles.

As in all other processes mentioned above, a subsequent step of classifying the resulting particles using a sieve in order to obtain a more uniform particle size distribution may be useful. Where necessary or useful, the particles may be dried at 25° C. under vacuum prior to classifying them.

When aiming for larger particles of the solid phase according to the invention, such as flakes or chips, the melt granulation process may also be modified in that the powder of the polysaccharide component is mixed into an amount of the fatty acid component large enough to allow the intimate mixture to be spread out or cast homogeneously onto a support, allowing it to solidify by cooling and/or drying and optionally breaking up the solidified material.

For instance, the flakes according to the invention are usually prepared by a process involving a casting step (i.e. pouring a liquid or semisolid material or mixture onto a support), followed by a step of breaking up the solidified cast material into flake shaped particles. The fatty acid glyceride component may be melted for this purpose, the polysaccharide component blended in and the mixture cast in a thin layer, for instance onto a conveyer belt or into dyes which are either cooled themselves and/or transported through, or past, a cooling section in order for the cast layer to solidify. The solidified cast layer may then be shaped into flake shaped particles by various means such as breaking, cutting or stencilling, optionally involving rotating means. In cases where the mixture is already cast into pre-shaped dyes, the shaping step is obviated and replaced by a step of merely removing the solidified, shaped casts from their dyes.

Alternatively, the intimate mixture may be cast onto a support in defined portions—for instance using a nozzle—the portions being selected such that the solidified cast material already has the desired size and shape. In this manner e.g. chips of the solid phase may be prepared, resembling the shape of Callets® as used in the confectionary field. Depending on the fluidity of the mixture upon being dosed from the nozzle or lack thereof, the portion of the material may also flatten to a more ‘button’ or ‘coin’ like shape, or it may solidify to droplets or cones respectively.

According to a further aspect of the invention, an ingestible particle is provided which is obtainable by the method(s) as described above.

In an alternative aspect of the invention, the water-soluble polysaccharide component in any of the preparation processes described above may be replaced by a water-insoluble, non-swelling, edible polysaccharide component, like e.g. cellulose, hemicellulose or long-chain and/or branched beta-glucans like curdlan, such as to obtain a solid phase comprising an intimate mixture of

(a) at least 10 wt.-% of a water-insoluble, non-swelling, edible polysaccharide component, and
(b) at least 10 wt.-% of a fatty acid glyceride component having a melting point of higher than 37° C., wherein the solid phase comprises not more than 5 wt.-% of mucoadhesive polymer, and/or wherein the combined content of the water-insoluble, non-swelling, edible polysaccharide component and of the fatty acid glyceride component in the solid phase is at least 80 wt.-%, and wherein the solid phase is provided in the form of
(i) at least one ingestible particle having a sieve diameter in the range from 0.01 mm to 10 mm, or from 0.01 mm to 20 mm; or
(ii) an ingestible non-particulate matrix.

In a further aspect, the invention provides a solid composition for oral administration comprising the solid phase as described above or which has been prepared from it, in the form of (a) a plurality of the particles as described above or (b) an ingestible non-particulate matrix as described above. In one embodiment, the solid composition has been prepared from a plurality of the particles, such as by compressing the particles into tablets.

Besides the solid phase of the invention, the compositions may comprise comestible goods, including ingestible liquids and solid goods such as cereals, corn flakes, nuts, seeds, dried fruits or vegetables, protein powders or granules, wafers, crispbread, rice puffs, as well as sugars, sugar alcohols, spices or the like. In case of ingestible liquids, these may either be provided in combination with the solid phase in a ready-to-use composition; or provided separately such that the consumer, or user, himself/herself adds it to the solid phase directly prior to ingestion. Optionally, the ingestible liquid and oral composition comprising the solid phase, or being prepared from it, may be provided in the form of a kit; i.e. both in separate packagings but distributed, or sold, in combination.

If not compressed into tablets, the solid phase in the form of particles may in principle be filled into capsules, sachets, stick packs, or containers (e.g. bottles or drink vials of glass or other materials). In one of the preferred embodiments, the particles, or granules, are filled into sachets, stick packs, or containers in such a way that a single dose is accommodated in one primary package. Optionally, the composition may comprise the particles along with one or more further inactive ingredients, such as e.g. colouring agents, stabilising agents, wetting agents, bulking agents, suspending agents, pH-modifiers, and/or flow-regulating agents. These particle based oral compositions may either be ingested as such or after suspending the particles in an ingestible liquid, such as water, juice, a dairy beverage such as milk or any other, preferably non-carbonated, ingestible liquid, or any mixture thereof.

If not filled into sachets, stick packs, capsules etc., the particles may also be provided in blends with comestible goods; e.g. flakes of the solid phase which are blended into a cereal composition, or muesli; or chips blended into ‘trail mix’ compositions or snack-bars. Optionally, ingestible liquids or semi-solids may be added to these compositions prior to consumption, e.g. milk to the flakes/cereal blend or yoghurt added to sprinkles.

Further alternatively, as described above, the solid composition for oral administration may also be provided with the solid phase being in the form of a non-particulate, ingestible matrix, for instance acting as a binder in ready-to-eat snack-bars. Here, the solid phase may either be blended with particulate comestible goods such as rolled oats, dried fruits, seeds or the like; or it may be applied onto and/or in between layers, or sheets, of comestible goods such as crispbread or wafers. In these embodiments, the solid phase is typically inseparably combined, or blended, with the comestible material(s) (unlike in the above embodiment of flakes being blended into cereal compositions where it would theoretically be possible to manually remove the solid phase flakes).

In one embodiment, a snack-bar, or muesli-bar, is formed by blending a melt of the solid phase with particulate comestible goods such as cereals (rolled oats, spelt flakes, corn flakes etc.), nuts (optionally, flaked or slivered), dried fruits, seeds, spices, aroma, protein powders or granules or similar components of snack-or muesli-bars. This solid phase/comestible goods-blend is spread on a surface such as a band-conveyor—optionally with the help of a casting-or spreading tool and/or with the help of a compaction tool—where the blend is allowed to solidify. The solidified blend may then either be cut into snack-or muesli-bar sized portions (commonly, cuboids of about 6 to 12 cm length, about 1 to 3 cm width and about 0.5 to 2 cm thickness); or broken up into ‘crunchier’ of bite-size or smaller. In an alternative embodiment, the solid phase/comestible goods-blend is spread on a structured surface comprising snack-or muesli-bar sized wells, such that after solidification no further cutting step is required and snack-or muesli-bar sized portions are obtained by simply extracting, or emptying, the solidified blend from the wells.

The molten solid phase may be the sole binder in these snack-or muesli bar compositions. Optionally, further binders may be added to the blend in order to either complement the binding action of the solid phase and/or to modify the test. Examples of suitable binders to be used in addition to the solid phase include syrups such as rice syrup, corn syrup as well as honey.

Optionally, the blend may be subjected to a further heating step prior to cutting or breaking up the solidified mass; for instance, passing the blend through a heating tunnel. This may be advisable e.g. when using rice syrup as an additional binder the blend.

It should be understood, that the solid phase may not only be incorporated into such snack-or muesli bars or ‘crunchier’ in the form of a non-particulate matrix (i.e. as a binder), but also in particulate form. For instance, the solid phase may be provided in the form of flakes, chips, pellets, or shavings which are blended at room temperature prior to adding e.g. rice syrup as the binder. This blend is then spread, optionally heated and shaped into bars and/or ‘crunchier’ as described above.

The presentation and oral administration in the form of particles in sachets, stick packs or containers (e.g. bottles or drink vials) or in the form of blends with comestible goods is also useful as it is preferred that a relatively large amount of the composition is administered as a single dose. In one of the preferred embodiments, a single dose unit or package comprises at least about 2 g of the composition, or at least about 3 g thereof. In another embodiment, a single dose unit or package comprises from about 3 g to about 100 g, or from about 3 g to about 20 g of the composition. It should be understood that these weights refer to the single dose unit or package as provided, or sold, to the consumer; for instance excluding the weight of any liquids which are not present in the single dose unit or package during shipping and storage but which may be added directly prior to actual ingestion by the user, or consumer (like milk being added to a single serving cup of muesli or juice being added to a dry pellet formulation in a drink vial package to form a drinkable suspension).

It should further be understood that the provision of single dose units or packages and their weights is not intended to exclude the option of multiple dose units or packages. The oral composition may also be provided in larger packages containing multiple doses together with instructions on obtaining a single dose; for instance a 350 g package containing a muesli mixture blended with the flakes of the invention with a serving suggestion printed on the side of the package, such as ‘Single serving about 70 g+add milk’.

The larger weight range (3 g to 100 g) commonly applies for compositions in which the solid phase is blended into snack-sized, or single serving sized, portions with comestible goods such as cereal flakes, rolled oats, seeds, crisp bread and the like (as described above). For instance, for the muesli composition comprising the solid phase according to the invention in the form of flakes, a single dose unit or package may comprise from about 3 g to about 100 g; e.g. from about 10 g to about 90 g, or from about 15 g to about 85 g, such as 15 g or 20 g or 40 g or 70 g.

In further embodiments, the amount comprised in a single dose is from about 4 g to about 15 g of the composition, or from about 5 g to about 12 g, or from about 5 g to about 10 g, respectively. The smaller weight range (3 g to 20 g) commonly applies for compositions in which the solid phase is the predominant weight fraction of the composition; for instance a plurality of particles provided in a stick pack.

The amount of the fatty acid glyceride component in the composition is at least 1 g, preferably at least 2 g. In a specific embodiment, a single dose unit or package comprises from about 3 g to about 100 g, or from about 3 g to about 20 g of the composition and the amount of the fatty acid glyceride component in the composition is preferably at least 2 g.

It is also preferred that the composition exhibits a high contents of the particles of the invention, such as at least about 20 wt.-%, or at least about 30 wt.-%, such as from about 20 wt.-% to 100 wt.-%, or from about 30 wt.-% to about 90 wt.-%. It is easily understood, that the specific weight fraction must be selected with regard to the specific composition; for instance, for solid phase pellets provided in a stick pack whose contents are supposed to be suspended in an ingestible liquid the weight fraction of pellets may be higher (e.g. in the range of about 60 wt.-% to about 100 wt.-%) than for a blend of solid phase flakes with cereals.

For the purpose of administration, the composition—more specifically the oral compositions comprising the solid phase of the invention in the form of particles (e.g. pellets)—may be suspended in a liquid or semisolid vehicle as mentioned above. The liquid may simply be water or fruit juice or a dairy beverage such as milk or any other, preferably non-carbonated, ingestible liquid, or mixtures thereof. As used herein, the term milk comprises milk-varieties obtained from animals (e.g. cow- , goat- or sheep milk) as well as milk varieties of vegetable/plant origin (e.g. soy- , rice- or nut based milks). The liquid(s) may optionally be provided together with the composition within a kit; e.g. both in separate primary packagings but distributed, or sold, in combination, such that the consumer, or user, himself/herself adds it to the solid phase directly prior to ingestion. This has the advantage that the nature and amount of liquid are controlled and the administration is more reproducible. Alternatively, the ingestible liquid may be provided in the same primary packaging as the ingestible particles, e.g. a drink vial or bottle, in the form of a ‘ready-to-use’ drink suspension, which does not require reconstitution by the consumer, or user, prior to ingestion. The reconstituted or ‘ready-to-use’ drink suspension may have, for example, a volume in the range from about 30 mL to about 300 mL, or from about 40 mL to about 250 mL, or from about 50 mL to about 200 mL.

In a preferred embodiment, the composition of the invention is administered as a suspension drink. It was found that the suspension drink of the invention is useful for administering large amounts, such as 1 g or more, and more typically at least 5 g, such as from about 10 g to about 100 g, of the composition while exhibiting good drinkability and mouth feel. Drinkability of such a suspension drink according to the invention may be assessed by methods used to determine the flowability of wet granular materials. In particular, dynamic measurements of the angle of repose may be taken using a rotating drum apparatus where the whole drum or its bottom and top are transparent or semi-transparent. Such apparatus are commercially available for instance from Mercury Scientific, USA (Revolution Powder Analyzer) and APTIS, Belgium (GranuDruM powder rheometer). In a suitable experimental set up for dynamic measurements of angle of repose of wet granular material comprising aqueous liquid, the drum is preferably made of PTFE (Teflon®) or coated with PTFE or similar anti-adhesive material, and is filled to half of its volume with a suspension of powder or particles. After placing the drum's top and bottom along a horizontal axis, and repeated tapping for even distribution of the drum's contents, the suspension forms a horizontal meniscus of an angle of zero. This may be visually observed and measured by standard methods of angle measurements. Rotating the drum along this horizontal axis may displace the meniscus of the powder suspension to a certain angle before the meniscus of the suspension repositions itself to an angle of almost zero. The displacement of the meniscus from the horizontal may be repeated several times, and a mean value of the dynamic angle of repose may be calculated.

In one embodiment, the suspension drink comprises a plurality of the particles of the invention and at least one aqueous liquid, and the sum of the volume fractions of the particles and the at least one aqueous liquid makes 100 vol-%. Accordingly, the present invention provides a suspension drink, comprising 50 to 75 vol-% of particles according to the invention; and 25 to 50 vol-% of at least one aqueous liquid; wherein the volume fractions are based on the total volume of the suspension drink. For reasons such as ease of suspending and swallowing, spherical spheroidal particles, e.g. pellets, with a mass median sieve diameter in the range from 0.01 mm to 10 mm are particularly preferred for such a suspension drink composition. Preferably, the dynamic angle of repose of the suspension drink is less than about 30°.

In a further preferred embodiment, the amounts of particles and liquid are selected such that a densely packed suspension drink is obtained by matching the filling height of the particles settled in a suitably sized container with the filling height of the aqueous liquid in the same container comprising the settled particles. In other words, the amount of the liquid is chosen in such manner that the meniscus of the liquid is roughly at the position of the upper limit of the settled particles.

The at least one aqueous liquid further may comprise alcohol, flavouring compounds, colouring compounds, preservatives, viscosity enhancers, health ingredients or mixtures of two or more thereof. Suitable flavouring compounds are citric acid, malic acid, phosphoric acid, tartaric acid, natural and synthetic aroma, sweeteners, for example monosaccharides, disaccharides, polyhydric alcohols; including arabitol, erythritol, glycerol, isomalt, lactitol, maltitol, mannitol, sorbitol or xylitol; or sugar substitutes, including cyclamate, saccharine, stevia, sucralose and/or aspartame. Further suitable flavouring compounds are juices of fruits and/or vegetables. Colouring compounds suitable for the aqueous liquid are for example Allura Red AC, Anthocyanine, azorubine, betanin, Brilliant Blue FCF, carotene, Quinoline Yellow WS, Ponceau 4R, Green S, Patent Blue V and tartrazine, either as such or in the form of the corresponding aluminium lakes. Suitable preservatives are vitamins A, E or C, retinyl palmitate, cysteine, methionine, citric acid, sodium citrate, used in amounts of 0.001 to 0.1% by weight based on the liquid.

The amount of the fatty acid glyceride component, which is a key ingredient of the composition, should preferably be at least about 1 g per single dose unit or package. In another embodiment, a single dose unit comprises at least about 2 g of the fatty acid glyceride component, such as about 3 g or about 4 g. In a further preferred embodiment, the content of the fatty acid glyceride component per single dose is at least about 5 g.

If present, the amount of the amino acid (or of the total amino acids, if a mixture or combination of amino acids is used) may be about 0.05 g or more per single dose unit or package. In another embodiment, a single dose unit comprises at least about 0.1 g, or at least about 0.2 g, or at least about 0.5 g of amino acid(s), respectively. In further embodiments, the content of the amino acid(s) per single dose unit is from 0.5 g to about 5 g, or from 0.5 g to about 3 g.

In one of the embodiments, the components of the particles, in particular the spherical spheroidal particles, such as pellets, with a mass median sieve diameter in the range from 0.01 mm to 10 mm, are selected such that the dynamic angle of repose of a suspension prepared from suspending the composition in water at a weight ratio of 1 is less than 30°.

As mentioned, the solid phase (in the form of particles or in matrix-form) and the oral compositions of the invention (comprising the solid phase or being prepared from it) may be used for the suppression of appetite, in particular in human subjects, and for the induction of satiety. This is equally valid for both the solid phase with the water-soluble polysaccharide component as defined herein and the solid phase with the water-insoluble, non-swelling, edible polysaccharide component as defined herein. Without wishing to be bound by theory, it is currently believed by the inventors that the appetite suppressing effect is at least in part based on the fatty acid compound comprised in the fatty acid glyceride component, which upon ingestion interacts with physiological targets located at/in the mucosa of the gastrointestinal tract, such as in the stomach and/or duodenum, thereby activating one or more signalling cascades which eventually produce a perception of satiety or a reduction of appetite or hunger. Possibly, one of the targets at which the one or more fatty acid(s) act are the ghrelin cells (or ghrelin receptors), large numbers of which are located in the stomach and the duodenum.

If present, amino acid(s) may further contribute to the appetite suppressing effect, which may be due to a stimulation of chemosensors in the proximal gastrointestinal tract by which in turn the CCK and glucagon secretion is triggered.

The polysaccharide component was found by the inventors to enhance the effect of the lipid which may possibly be due to the prolonged integrity of the particles and/or masticated pieces of the solid phase, allowing for an increased interaction of the fatty acid(s) of the glyceride with the target structure and resulting in an increased bioavailability of the fatty acid glyceride component(s), as for instance measured by increased cPDR in a breath test study (see Example 2). Of course, other properties of the solid phase may also effect or contribute to the prolonged integrity of the solid phase particles and/or the masticated pieces of the solid phase, and the increased bioavailability of the fatty acid glyceride component(s), such as the selected particle size or the low density resulting from the high lipid content. Without wishing to be bound by theory, the increase in bioavailability may e.g. be caused by the prolonged integrity of the solid phase according to the invention which may result in more rapid gastric emptying of the solid phase particle(s) and/or the masticated pieces of the solid phase.

In any case, the inventors found that the oral administration of the solid phase (in form of particles or in form of a non-particulate matrix) to volunteers induced satiety with the consequence that the subjects experienced suppressed appetite and showed a reduced food intake during the meal following the administration of a composition comprising the particles as described herein. This effect was consistent with animal data showing the composition leads to a weight loss, or weight reduction, of the test animals.

The solid phase and/or compositions of the invention may therefore be used clinically, or as dietary supplements, for the prevention and/or treatment of obesity and overweight, as well as the prevention and/or treatment of diseases or conditions associated with obesity; e.g. by using the ingestible solid phase as defined herein and/or compositions comprising or prepared from the solid phase (for instance from a plurality of solid phase particles)for body weight reduction.

As said, one aspect of the invention provides a method for the prevention and/or treatment of obesity and overweight, as well as the prevention and/or treatment of diseases or conditions associated with obesity, for appetite suppression, body weight reduction and/or for the induction of satiety, said method comprising a step of orally administering the solid phase of the invention (in the form of particles or in the form of a non-particulate matrix) and/or compositions comprising or prepared from said solid phase (for instance from a plurality of solid phase particles). Optionally said method comprises the oral administration of the solid phase and/or compositions at least once a day over a period of at least one week.

In yet other words, one aspect of the invention provides the use of the solid phase of the invention (in the form of particles or in the form of a non-particulate matrix) and/or compositions comprising or prepared from said solid phase (for instance from a plurality of solid phase particles) in the manufacture of medicaments for the prevention and/or treatment of obesity and overweight, as well as the prevention and/or treatment of diseases or conditions associated with obesity, for appetite suppression, body weight reduction and/or for the induction of satiety. Optionally, this comprises the oral administration of the solid phase and/or compositions at least once a day over a period of at least one week.

As used herein, obesity is a medical condition in which excess body fat has accumulated to the extent that it may have an adverse effect on health. Overweight is understood as a borderline condition characterised by a body mass index (BMI) between 25 and below 30. Starting from a BMI of 30, the condition is classified as obesity.

In one embodiment, the particles and/or compositions are administered to normal weight or overweight subjects gaining weight over time or otherwise being at risk of developing obesity. In this case, the therapeutic objective is to stop or limit the weight gain and prevent the development of obesity. Another purpose may be to reduce the risk that the subject develops a disease or condition associated with or caused by obesity.

In a further embodiment, the solid phase (in the form of particles or in the form of a non-particulate matrix) and/or compositions are administered to obese patients in order to treat or reduce the severity of obesity. Again, the therapeutic use may also be directed to the reduction of the risk of developing a disease or condition associated with or caused by obesity.

A large number of diseases and conditions are nowadays considered to be associated with or caused by obesity, even though the mechanism by which they are linked to obesity may not always be fully understood. In particular, these diseases and conditions include—without limitation—diabetes mellitus type 2, arterial hypertension, metabolic syndrome, insulin resistance, hypercholesterolaemia, hypertriglyceridaemia, osteoarthritis, obstructive sleep apnoea, ischaemic heart disease, myocardial infarction, congestive heart failure, stroke, gout, and low back pain. The prevention and/or reduction of risk for developing any of these conditions falls within the scope of the therapeutic use according to the invention.

Moreover, the therapeutic use preferably involves the at least once daily oral administration of the solid phase (in the form of particles or in the form of a non-particulate matrix) and/or compositions of the invention over a period of at least one week. In this context, the expression “therapeutic use” is understood to also cover the preventive or prophylactic use. In a further preferred embodiment, the solid phase and/or compositions are administered to a human subject over a period of at least about 2 weeks, or at least about 4 weeks, or at least about 6 weeks, or at least about 2 months, respectively. Also preferred is an administration regimen providing for once or twice daily administration.

The time of administration should be selected to maximise the satiety-inducing effect on the amount of food which is subsequently taken up by the subject that is treated. For example, it is useful to administer a dose of the composition before a major meal, such as before a lunchtime meal and/or before the evening dinner such as to reduce the amount of food eaten during either of these meals. With respect to the precise timing, it is preferred that the dose is administered within about 5 minutes to 120 minutes prior to the respective meal, in particular about 10 minutes to about 120 minutes prior to the meal, or about 15 minutes to about 90 minutes prior to the meal, such as about 30 minutes or about 60 minutes prior to the meal.

In one embodiment, a dose unit comprising at least about 5 g of the fatty acid glyceride component is administered to a human subject at least once daily between about 15 minutes and about 90 minutes prior to a meal over a period of at least 4 weeks for the prevention and/or treatment of obesity or an associated disease.

In yet another aspect of the invention provides the use of the solid phase of the invention as a taste-neutral binder for solid comestible goods; e.g. as a binder in sweet or savoury snacks formed from cereals, corn flakes, dried fruits or vegetables, seeds, nuts, protein powders or granules, rice puffs, soy puffs, crisp-bread, wafers, spices, sugar, sugar alcohols, or the like. The term ‘taste-neutral’ as used herein refers to materials which are neither exhibiting a distinct sweet, salty, sour, bitter nor umami taste sensation; i.e. materials whose faint inherent taste is commonly not or hardly noticeable anymore once blended with other taste-bearing materials.

Further potentially useful embodiments are easily derivable on the basis of the guidance provided herein-above and the following examples.

### EXAMPLES

### Example 1: Particle Integrity Assay

PromOat® beta-glucan was from Tate & Lyle, Sweden. Inulin was from Spinnrad, Germany. Benefiber® resistant dextrin (also known as Benefiber® Nutriose®) was from Novartis, UK. Nutriose® FB resistant dextrin and Nutralys® S85F (a pea protein) were obtained from Roquette, France. Unisol DP (a soy protein with a protein content of approx. 90%) was obtained from Vitablend, Netherlands.

Palm stearin and Omega-3 fat powder were from Bressmer, Germany. Prifex® 300 palm stearin was from Unimills, The Netherlands. Plant-derived Omega-3-Concentrate powder 67 and Omega-3-Concentrate oil based on linseed oil were from Bressmer, Germany. Safflower oil was from Brokelmann, Germany.

Granules were prepared by melting one lipid (e.g. at 50° C. to 70° C.) and optionally adding other lipid components and a few crystals of Oil Red O (Sigma Aldrich, USA) to obtain a homogenous melt or suspension. For test samples polymer(s) were incorporated by mechanical mixing. Each composition was transferred into a zip-loc-bag and cooled to −18° C. in a freezer. The material was first crushed by means of a hammer, shredded to a granulate in a kitchen blender (Bosch ProfiMIXX, Germany), optionally dried under vacuum at 25° C. and then classified through a set of wire mesh sieves (VWR International, Germany) to a granulate size of below 2.0 mm and above 1.3 mm.

Fresh pork stomach (from a local butcher) was cut into 3 cm×3 cm pieces and placed into the bottom of a glass petri dish (10 cm diameter). 22 mL fasted-state simulated gastric fluid (FaSSGF) were added to the petri dish. FaSSGF was prepared by dis-solving 1 g of NaCl (Sigma-Aldrich) in 450 mL of water, adding 30 mg of SIF powder (biorelevant.com), adjusting the pH to 2.0 with 0.1 N HCl (Sigma-Aldrich) and adding water to a final volume of 500 mL. The petri dish was covered and placed onto a petri dish shaker (ST5 from CAT, Germany) set to a tilt angle of 12° and a speed of 50/min. The shaker was placed into an oven heated to a temperature of 37° C. After 30 minutes, 350 mg granulate were added to the contents of the petri dish without interrupting agitation. After 5 min, the samples were removed from the oven, and the piece of pork stomach was rinsed three times with water (3 mL each). Any material bound to the stomach surface was removed by means of a spatula, transferred into a weighing dish, and dried to constant weight (electronic moisture meter MLB 50-3N, Kern & Sohn, Germany). This dry weight, which at least in parts reflects the weight of potentially present mucoadhesive material, was recorded and calculated as percent of initial granulate weight, representing binding as a measure of mucoadhesiveness.

For determining the particle integrity, the petri dish containing the remaining unbound material was agitated at 37° C. for another 15 min, and particle integrity was classified by visual inspection as “low” (complete disintegration or disintegration of at least 50% of the particles), or “high” (disintegration of less than 50% of the particles) or “medium” (disintegration of less than 50% of the particles, but visible loss of small amounts of powders from the particles).

In result, it was found that most test compositions with particles according to the invention showed high particle integrity with no or almost no mucoadhesion, as shown in the table below.

| Sample | Glyceride (g) | Polysaccharide (g) | Binding | Integrity |
|---|---|---|---|---|
| Test 1 | Palm stearin, 5 g | Benefiber, 5 g | 0% | high |
| Test 2 | Prifex 300, 5 g | Benefiber, 5 g | 0% | high |
| Test 3 | Prifex 300, 4 g | Benefiber, 6 g | 0% | high |
| | Safflower oil, 2 g | | | |
| Test 4 | Palm stearin, 5 g | Inulin, 5 g | n.d. | high |
| Test 5 | Palm stearin, 5 g | Benefiber, 2 g | n.d. | medium |
| | | PromOat, 2 g | | |
| Test 6 | Prifex 300, 6 g | PromOat, 5 g | 0% | high |
| Test 7 | Prifex 300, 4 g | Konjac glucomannan, 1 g | 0% | medium |
| | Safflower oil, 2 g | PromOat, 4 g | | |
| Test 8 | Prifex 300, 6 g | Konjac glucomannan, 1 g | 0% | high |
| | | Benefiber, 2 g | | |
| | | PromOat, 2 g | | |
| Test 9 | Prifex 300, 4 g | Benefiber, 3 g | 0% | high |
| | Omega-3-Concentrate | | | |
| | powder 67, 3 g | | | |
| Test 10 | Prifex 300, 4 g | Benefiber, 4.5 g | 0% | high |
| | Safflower oil, 1 g | | | |
| | Omega-3-Concentrate | | | |
| | powder 67, 1.5 g | | | |
| Test 11 | Prifex 300, 6 g | Konjac glucomannan, 1 g | 0% | high |
| | | Benefiber, 2 g | | |
| Test 12 | Prifex 300, 9 g | Nutriose FB, 10 g | n.d. | high |
| | Omega-3-Concentrate oil, | | | |
| | 1 g | | | |
| Test 13 | Prifex 300, 7 g | Nutriose FB, 10 g | n.d. | high |
| | Safflower oil, 2 g | | | |
| | Omega-3-Concentrate oil, | | | |
| | 1 g | | | |
| Test 14 | Prifex 300, 8 g | Nutriose FB, 10 g | n.d. | high |
| | Omega-3-Concentrate oil, | | | |
| | 2 g | | | |
| Test 15 | Gelucire 43/10, 10 g | Nutriose FB, 10 g | n.d. | high |
| Test 16 | Prifex 300, 10 g | Nutralys S85F, 5 g | n.d. | medium |
| | | Nutriose FB, 5 g | | |
| Test 17 | Prifex 300, 10 g | Nutralys S85F, 4 g | n.d. | medium |
| | | Nutriose FB, 6 g | | |
| Test 18 | Prifex 300, 10 g | Nutralys S85F, 3 g | n.d. | medium |
| | | Nutriose FB, 7 g | | |
| Test 19 | Prifex 300, 10 g | Unisol DP, 5 g | n.d. | high |
| | | Nutriose FB, 5 g | | |
| Test 20 | Prifex 300, 10 g | Unisol DP, 4 g | n.d. | high |
| | | Nutriose FB, 6 g | | |
| Test 21 | Prifex 300, 9 g | Unisol DP, 5 g | n.d. | medium |
| | Omega-3 oil, 1 g | Nutriose FB, 5 g | | |

### Example 2: Breath Tests on Healthy Volunteers

Gastrointestinal half-life and bioavailability of free fatty acids were assessed using the ¹³C-octanoic acid breath test. The labelled octanoic acid substrate is rapidly absorbed in the intestine and metabolised in the liver with the production of ¹³CO₂, which is exhaled, thus reflecting uptake of octanoic acid from the gastrointestinal tract and after exit from the stomach. At the beginning of the experiment a reference breath sample was taken from the subject. Subsequently, the subject consumed a load of either lipid granulate as reference sample, or lipid granulate containing the polysaccharides as test sample.

Prifex® 300 palm stearin was from Unimills, The Netherlands. Benefiber® resistant dextrin (also known as Benefiber® Nutriose®) was from Novartis, UK.

Granulate was prepared by melting lipid at 50° C. and adding 100 mg of ¹³C octanoic acid (Campro Scientific, The Netherlands), and—for test samples—incorporating polymer.

The mixture was subsequently transferred into a zip-loc-bag and cooled to −18° C. in a freezer. The material was crushed by means of a hammer, shredded to a granulate in a kitchen blender (Bosch, Germany), dried under vacuum at 25° C. and classified through a set of wire mesh sieves (VWR International, Germany) to a granulate size of below 1.3 mm and above 0.5 mm.

For sample ingestion, frozen granulate was mixed with 100 g cold yogurt (fruit flavour, ca. 100 calories) and consumed within one to two minutes. After ingesting the samples, subject exhaled through a mouthpiece to collect an end-expiratory breath sample into a 300 mL foil bag at time intervals. Breath samples were taken over a period of 410 min. During this time period, 0.5-1.0 L of water were drunk at a rate of approximately one glass per hour, a light lunch was consumed after 180 min, and physical exercise represented daily routine.

After completion of breath bag collection, analysis was performed by means of a FANci2 breath test analyser based on non-dispersive infrared spectroscopy (Fischer Analysen Instrumente GmbH, Germany). ¹³C abundance in breath was expressed as relative difference (‰) from the universal reference standard (carbon from Pee Dee Belemnite limestone). ¹³C enrichment was defined as the difference between ¹³C abundance in breath prior to sample ingestion and ¹³C abundance at the defined time points after sample ingestion and was given in delta over basal (DOB, ‰). From the breath test analyser's operating software (FANci version 2.12.42.14 02/14), values of cumulated percent dose rate (cPDR, corresponding to bioavailability) were taken to protocol.

As shown in the table below, it was found that the particles of test composition 1 lead to an increase in bioavailability of the fatty acid, as measured by the breath test (see cPDR), which effect is due to the combination of the glyceride component with the resistant dextrin. This in turn has resulted in increased satiety in human volunteers.

| Sample | Fatty acid glyceride | Polysaccharide | cPDR (%) |
|---|---|---|---|
| Reference 1 | Prifex 300: 6 g | — | 29.0 |
| Test composition 1 | Prifex 300: 6 g | Benefiber: 6 g | 78.2 |

### Example 3: Binders for Food Products

A mixture of palm stearin (Prifex® 300 from Unimills, The Netherlands), resistant dextrin (Nutriose® FB 06, Roquette, France) and soy protein concentrate (Unisol DP IP or HS90 IP, Vitablend, The Netherlands) was used as binder for the production of bars and cereal snacks. Safflower oil or linseed oil (Bressmer & Francke, Germany) were added optionally.

Binders 1-5 were prepared by melting palm stearin at 60° C., optionally together with plant oil. Soy protein concentrate was added and stirred to obtain a homogenous melt. Nutriose was added and mixed thoroughly. Binders were used immediately or after storage. For storage, each molten binder was transferred into zip-loc-bags and cooled to −18° C. in a freezer. The frozen material was first crushed by means of a hammer and then shredded to a granulate in a kitchen blender (Bosch ProfiMIXX, Germany or Vitamix®, USA) and stored at room temperature. For use after storage the binder granulate was brought to a melt at 60° C.

Binder 1:

100 g palm stearin, 50 g soy protein concentrate, 50 g resistant dextrin.

Binder 2:

75 g palm stearin, 15 g safflower oil, 45 g soy protein concentrate, 45 g resistant dextrin.

Binder 3:

100 g palm stearin, 40 g soy protein concentrate (HS90 IP), 50 g resistant dextrin.

Binder 4:

90 g palm stearin, 10 g linseed oil, 50 g soy protein concentrate, 50 g resistant dextrin.

Binder 5:

70 g palm stearin, 20 g safflower oil, 70 g soy protein concentrate, 50 g resistant dextrin.

### Example 3.1: Crispy Bars

Cereal bars were created using binders 1-5, soy protein nuggets (Supro® Plus Nuggets 60, DuPont, USA) grain flakes, rice syrup, dried fruits and optionally chocolate. 21 g binder was melted in a pot at 60° C. to obtain a homogenous melt. 10 g soy protein nuggets (Supro® Plus Nuggets 60, DuPont, USA) and 5 g spelt flakes (Albtal Naturkost, Germany) together with 8 g dried cranberries (K-Klassik, Germany) were mixed directly in the pot with the binder. 10 g rice syrup and, if requested, 5 g chocolate was added, quickly mixed and transferred in a silicon baking dish (Silikomart®, Italy) to create two bars. The bars were heated in the oven for 3 minutes at 180° C. with circulating air. After cooling down completely at room temperature bars could be removed from the baking dish.

### Example 3.2: Crispy Bar with Chocolate

Cereal bars were created using binders 1-5, soy protein nuggets (Supro® Plus Nuggets 60, DuPont, USA) grain flakes, rice syrup, dried fruits and optionally chocolate. 21 g binder was melted in a pot at 60° C. to obtain a homogenous melt. 10 g soy protein nuggets (Supro® Plus Nuggets 60, DuPont, USA) and 5 g spelt flakes (Albtal Naturkost, Germany) together with 8 g dried cranberries (K-Klassik, Germany) were mixed directly in the pot with the binder. 10 g rice sirup and, if requested, 5 g chocolate was added, quickly mixed and transferred in a silicon baking dish (Silikomart®, Italy) to create two bars. The bars were heated in the oven for 3 minutes at 180° C. with circulating air. After cooling down completely at room temperature bars could be removed from the baking dish.

### Example 3.3: Crunchy Snacks

Cereal snacks were created using binders 1-5, soy protein nuggets (Supro® Plus Nuggets 60, DuPont, USA) grain flakes, rice syrup, and dried fruits. 40 g binder was melted in a pot at 60° C. to obtain a homogenous melt. 20 g soy protein nuggets (Supro® Plus Nuggets 60, DuPont, USA) and 20 g spelt flakes (Albtal Naturkost, Germany) together with 15 g dried cherries (Seeberger, Germany) were mixed directly in the pot with the binder. 20 g rice syrup were added, quickly mixed and transferred on a baking tray to create a layer of approx. 1 centimeter thickness. The mixture was heated in a circulating air oven for 3 minutes at 180° C. After cooling to room temperature the layer was broken in pieces by means of a knife.

### Example 3.4: Crunchy Snacks with Chocolate

Cereal snacks were created using binders 1-5, soy protein nuggets (Supro® Plus Nuggets 60, DuPont, USA) grain flakes, rice sirup, and dried fruits and cocoa powder and chocolate. 40 g binder was melted in a pot at 60° C. to obtain a homogenous melt. 20 g soy protein nuggets (Supro® Plus Nuggets 60, DuPont, USA) and 20 g spelt flakes (Albtal Naturkost, Germany) together with 15 g dried cherries (Seeberger, Germany) and 1 g cocoa powder (Stollwerk, Germany) were mixed directly in the pot with the binder. 20 g rice syrup and 8 g chocolate were added, quickly mixed and transferred on a baking tray to create a layer of approx. 1 centimeter thickness. The mixture was heated in a circulating air oven for 3 minutes at 180° C. After cooling to room temperature the layer was broken in pieces by means of a knife.

### Example 4: Cereal Containing Lipid Flakes

25 kg palm stearin (Prifex® 300, Brenntag B.V. Belgium) were filled into a ploughshare batch mixer (FM130, Gebrüder Lödige Maschinenbau GmbH, Germany). The chamber was heated using an external temperature control system (Compact TKN-90-18-35, Single Temperiertechnik GmbH, Germany) with the mixing tool running at 30 rpm. After the lipid components were brought to a homogeneous melt at 60° C., 12.5 kg soy protein (Unisol DP IP Non GMO, Barentz, Netherlands) were added and blended at 40 rpm until homogeneity. Then 12.5 kg resistant dextrin (Nutriose® FM06, Barentz, Netherlands) were added and again blended at 40 rpm. After mixing was completed the heating system was turned off and 12 kg of dry ice were rapidly introduced into the mixing chamber with the mixer running at 40 rpm.

Subsequently, the milling head (speed 2) was activated and granulate premix was obtained, released through the outlet and collected in bags. The obtained premix was molten in an external heating vessel at 60° C. and spread on a conveyer belt to give a thickness of approx. 1 mm. The spread was cut into strands of 10 mm. Subsequently the conveyer belt was run through a cooling channel and the material brought to room temperature. At the end of the conveyer belt the solidified strands were broken into squares or rectangle by means of rotating blades and collected into paper bags. A breakfast cereal was created by mixing 30 g of lipid flakes with 20 g of oat flakes, 30 g of oven roasted spelt flakes and 10 g of dried sweetened cranberries.

1. An ingestible solid phase comprising an intimate mixture of
(a) at least 10 wt.-% of a water-soluble polysaccharide component based on glucose or fructose having an average degree of polymerisation from 2 to 100, or from 4 to 80, and
(b) at least 10 wt.-% of a fatty acid glyceride component having a melting point of higher than 37° C.,
wherein the solid phase comprises not more than 5 wt.-% of mucoadhesive polymer, and
wherein the solid phase is provided in the form of
(i) at least one ingestible particle having a sieve diameter in the range from 0.01 mm to 10 mm or from 0.01 mm to 20 mm; or
(ii) an ingestible non-particulate matrix.

2. An ingestible solid phase comprising an intimate mixture of
(a) at least 10 wt.-% of a water-soluble polysaccharide component based on glucose or fructose having an average degree of polymerisation from 2 to 100, or from 4 to 80, and
(b) at least 10 wt.-% of a fatty acid glyceride component having a melting point of higher than 37° C.,
wherein the combined content of the water-soluble polysaccharide component and of the fatty acid glyceride component in the solid phase is at least 80 wt.-%; and wherein the solid phase is provided in the form of
(i) at least one ingestible particle having a sieve diameter in the range from 0.01 mm to 10 mm, or from 0.01 mm to 20 mm; or
(ii) an ingestible non-particulate matrix.

3. The solid phase of claim 1, being substantially free of mucoadhesive polymer.

4. The solid phase of claim 1, wherein the polysaccharide component
(a) exhibits a solubility of at least 2 wt.-%, and optionally of at least 5 wt.-%, measured in purified water at 25° C.; and/or
(b) comprises a neutral polysaccharide that is resistant to digestion in the human small intestine.

5. The solid phase of claim 1, wherein the polysaccharide component comprises
(a) a dextrin having a degree of polymerisation from 4 to 40, or from 10 to 30, preferably from 12 to 25; and/or
(b) an inulin having a degree of polymerisation from 4 to 60, or from 5 to 25.

6. The solid phase of claim 1, wherein the fatty acid glyceride component
(a) has a melting point from 38° C. to 75° C., in particular from 40° C. to 70° C.; and/or
(b) comprises fatty acid triglycerides, wherein the content of the fatty acid triglyceride in the solid phase is at least 10 wt.-%.

7. The solid phase of claim 1, wherein the polysaccharide component comprises a resistant dextrin derived from wheat or maize starch, and wherein the fatty acid glyceride component comprises a fractionated but non-hydrogenated palm stearin or palm kernel stearin.

8. The solid phase of claim 1, being in the form of an ingestible particle, wherein the particle is provided as a granule, a pellet, a minitablet, a flake, a shaving, a chip, a sprinkle or mixtures thereof.

9. A method for the preparation of the solid phase according to claim 1, comprising the steps of
(i) preparing an intimate mixture comprising the water-soluble polysaccharide component and the fatty acid glyceride component by melting the fatty acid glyceride component at least partially and blending in the polysaccharide component, and
(ii) applying the intimate mixture onto comestible goods prior to solidification;
(iii) blending the intimate mixture with comestible goods prior to solidification; or
(iv) processing the intimate mixture further to obtain ingestible particles of said mixture by
(a) extruding the mixture using a screw extruder;
(b) spray congealing the mixture, optionally using a jet-break-up technique;
(c) melt granulating the mixture;
(d) compressing the mixture into minitablets;
(e) melt injection of the mixture into a liquid medium;
(f) spray coating of the mixture onto inert cores.

10. (canceled)

11. A solid composition for oral administration comprising the solid phase of claim 1 in the form of
(a) a plurality of particles, wherein the mass median sieve diameter of the particles is in the range from 0.01 mm to 20 mm, or from 0.01 mm to 10 mm, in particular from 0.1 mm to 3 mm, or
(b) an ingestible non-particulate matrix.

12. A single dose unit or package comprising the composition of claim 11, wherein the amount of the composition is from 3 g to 100 g, or from 3 g to 20 g, and/or wherein the amount of the fatty acid glyceride component in the composition is at least 2 g.

13. The composition of claim 11 for use in
(a) the prevention and/or treatment of obesity or a disease or condition associated with obesity,
(b) appetite suppression,
(c) induction of satiety, and/or
(d) body weight reduction;
wherein the use optionally comprises the oral administration of the composition at least once a day over a period of at least one week.

14. An ingestible solid phase comprising an intimate mixture of
(a) at least 10 wt.-% of a water-insoluble, non-swelling, edible polysaccharide component, and
(b) at least 10 wt.-% of a fatty acid glyceride component having a melting point of higher than 37° C.,
wherein the solid phase comprises not more than 5 wt.-% of mucoadhesive polymer, and/or
wherein the combined content of the water-insoluble, non-swelling, edible polysaccharide component and of the fatty acid glyceride component in the solid phase is at least 80 wt.-%; and
wherein the solid phase is provided in the form of
(i) at least one ingestible particle having a sieve diameter in the range from 0.01 mm to 10 mm, or from 0.01 mm to 20 mm; or
(ii) an ingestible non-particulate matrix.

15. A method for the preparation of the solid phase according to claim 14, comprising a step of
(i) preparing an intimate mixture comprising the water-insoluble, non-swelling, edible polysaccharide component and the fatty acid glyceride component by melting the fatty acid glyceride component at least partially and blending in the polysaccharide component, and
(ii) applying the intimate mixture onto comestible goods prior to solidification;
(iii) blending the intimate mixture with comestible goods prior to solidification; or
(iv) processing the intimate mixture further to obtain ingestible particles of said mixture by
(a) extruding the mixture using a screw extruder;
(b) spray congealing the mixture, optionally using a jet-break-up technique;
(c) melt granulating the mixture;
(d) compressing the mixture into minitablets;
(e) melt injection of the mixture into a liquid medium;
(f) spray coating of the mixture onto inert cores.

16. (canceled)

17. A solid composition for oral administration comprising the solid phase of claim 14 in the form of
(a) a plurality of particles, wherein the mass median sieve diameter of the particles is in the range from 0.01 mm to 20 mm, or from 0.01 mm to 10 mm, in particular from 0.1 mm to 3 mm, or
(b) an ingestible non-particulate matrix.

18. A single dose unit or package comprising the composition of claim 17, wherein the amount of the composition is from 3 g to 100 g, or from 3 g to 20 g, and/or wherein the amount of the fatty acid glyceride component in the composition is at least 2 g.

19. The composition of claim 17 for use in
(a) the prevention and/or treatment of obesity or a disease or condition associated with obesity,
(b) appetite suppression,
(c) induction of satiety, and/or
(d) body weight reduction;
wherein the use optionally comprises the oral administration of the composition at least once a day over a period of at least one week.
